(19) United States
(12) Patent Application Publication
(10) Pub. No.: US 2006/0154243 A1
(43) Pub. Date: Jul. 13, 2006

(54) Antigenic peptides of SARS coronavirus and uses thereof

(76) Inventors: Jan Ter Meulen, Marburg (DE); Jaap Goudsmit, Amsterdam (NL); Peter Timmerman, Lelystad (NL); Jelle Wouter Slootstra, Lelystad (NL)

Correspondence Address: TRASK BRITT, P.O. BOX 2550, SALT LAKE CITY, UT, 84110, US

(21) Appl. No.: 11/337,226

(22) Filed: Jan. 20, 2006

Related U.S. Application Data
(63) Continuation of application No. PCT/EP04/51548, filed on Jul. 20, 2004.

(30) Foreign Application Priority Data
Jul. 21, 2003 (WO) PCT/EP03/50324
Sep. 2, 2003 (WO) PCT/EP03/50393
Oct. 27, 2003 (WO) PCT/EP03/50762

(51) Int. Cl. C12Q 1/70; C07K 16/10; C07K 14/165

(52) U.S. Cl. 435/5; 530/350; 530/388.3

(57) ABSTRACT

The present invention pertains to antigenic peptides of SARS-CoV and their use in diagnostic test methods and in the treatment of condition resulting from SARS-CoV. Furthermore, this invention provides antibodies capable of specifically recognizing the peptides of the invention. The antibodies can also advantageously be used in diagnostic test methods and in the treatment of conditions resulting from SARS-CoV.

### CROSS-REFERENCE TO RELATED APPLICATIONS

This application is a continuation of PCT International Patent Application No. PCT/EP2004/051548, filed on Jul. 20, 2004, designating the United States of America, and published, in English, as PCT International Publication No. WO 2005/012338 A1 on Feb. 10, 2005, which application claims priority to International Patent Application No. PCT/EP03/050762 filed Oct. 27, 2003, which claims priority to International Patent Application No. PCT/EP03/50393 filed Sep. 2, 2003, which, in turn, claims priority to International Patent Application No. PCT/EP03/50324 filed Jul. 21, 2003, the contents of the entirety of each of which are incorporated by this reference.

### FIELD OF THE INVENTION

Various embodiments of the present invention generally relate to biotechnology. Further, various embodiments relate to medicine. In particular, the invention relates to antigenic peptides of SARS coronavirus and uses thereof.

### BACKGROUND OF THE INVENTION

Recently, a new and, in several cases, deadly clinical syndrome was observed in the human population, now called severe acute respiratory syndrome (SARS) (Holmes, 2003). The syndrome is caused by a novel coronavirus (Ksiazek et al., 2003), referred to as the SARS-CoV. The genome sequence of SARS-CoV has been determined (Rota et al., 2003; Marra et al., 2003). However, much remains to be learned about this virus, and means and methods for diagnostics and treatment of the virus and the syndrome are needed. The present invention provides means and methods for use in diagnostics, treatment and prevention of SARS-CoV.

### SUMMARY OF THE INVENTION

The present invention pertains to antigenic peptides of SARS-CoV. Furthermore, the invention provides fusion proteins comprising these peptides and antibodies against these peptides. The use of the peptides, fusion proteins and antibodies in the treatment of a condition resulting from SARS-CoV and a diagnostic test method for determining the presence of antibodies recognizing SARS-CoV in a sample or for determining the presence of SARS-CoV in a sample are also contemplated in the present invention.

### DETAILED DESCRIPTION OF THE INVENTION

In a first aspect, the invention provides antigenic peptides of SARS-CoV. In particular, the peptides found are peptides of (potential) proteins of the SARS-CoV strains called TOR2, Frankfurt 1 and HSR 1. In the present invention, binding of sera from SARS patients to a series of overlapping 15-mer peptides, which were either in linear form or in looped/cyclic form, of proteins from the SARS-CoV strains mentioned above was analyzed by means of PEPSCAN analysis (see, inter alia, WO 84/03564, WO 93/09872, Slootstra et al. 1996).

The complete genome and the amino acid sequence of the proteins and potential proteins of various known strains of SARS-CoV can be found in the EMBL-database and/or other databases. For instance, the complete genome of the SARS coronavirus Urbani can be found under accession number AY278741, the complete genome of the SARS coronavirus HSR 1 can be found under accession number AY323977, the complete genome of the SARS coronavirus Frankfurt 1 can be found under accession number AY291315 and the complete genome of the SARS coronavirus TOR2 can be found under accession number AY274119.

Several proteins of SARS-CoV, such as inter alia the spike protein, are shared by all SARS-CoV strains. However, the strains TOR2, Frankfurt 1 and HSR 1 contain open reading frames encoding (potential) proteins that are not present in the SARS-CoV strain Urbani. In the SARS-CoV strain called TOR2 these (potential) proteins are called Orf9, Orf10, Orf13 and Orf14. The first three of these (potential) proteins are also found in the SARS-CoV strains called Frankfurt 1 and HSR 1. In these strains the (potential) proteins are called Orf7b, Orf8a and Orf9b, respectively. The coding sequence (CDS) of the (potential) proteins of SARS-CoV TOR2 is shown under EMBL-database accession number AY274119, the coding sequence (CDS) of the (potential) proteins of SARS-CoV HSR 1 can be found under accession number AY323977, the coding sequence (CDS) of the (potential) proteins of SARS-CoV Frankfurt 1 can be found under accession number AY291315.

The present invention discloses antigenic peptides of the SARS-CoV TOR2 (potential) proteins called Orf9 (the protein-id of Orf9 is AAP41044, see also SEQ ID NO:1), Orf10 (the protein-id of Orf10 is AAP41045, see also SEQ ID NO:2), Orf13 (the protein-id of Orf13 is AAP41048, see also SEQ ID NO:3) and Orf14 (the protein-id of Orf14 is AAP41049, see also SEQ ID NO:4). The antigenic peptides of Orf9 of the strain TOR2 are also present in the (potential) protein called Orf7b of strain Frankfurt 1 (the protein-id of Orf7b is AAP33704, see also SEQ ID NO:5) and strain HSR 1 (the protein-id of Orf7b is AAP72981, see also SEQ ID NO:6). The antigenic peptides of Orf10 of the strain TOR2 are also present in the (potential) protein called Orf8a of strain Frankfurt 1 (the protein-id of Orf8a is AAP33705, see also SEQ ID NO:9) and strain HSR 1 (the protein-id of Orf8a is AAP72982, see also SEQ ID NO:10). The antigenic peptides of Orf13 of the strain TOR2 are also present in the (potential) protein called Orf9b of strain Frankfurt 1 (the protein-id of Orf9b is AAP33708, see also SEQ ID NO:7) and strain HSR 1 (the protein-id of Orf9b is AAP72985, see also SEQ ID NO:8).

In one embodiment, the invention provides a peptide having an amino acid sequence selected from the group consisting of LIMLIIFWFSLEIQD (SEQ ID NO:11), IMLIIFWFSLEIQDL (SEQ ID NO:12), MLIIFWFSLEIQDLE (SEQ ID NO:13), LIIFWFSLEIQDLEE (SEQ ID NO:14), IIFWFSLEIQDLEEP (SEQ ID NO:15), IFWFSLEIQDLEEPC (SEQ ID NO:16), FWFSLEIQDLEEPCT (SEQ ID NO:17), WFSLEIQDLEEPCTK (SEQ ID NO:18), FSLEIQDLEEPCTKV (SEQ ID NO:19), CSCICTVVQRCASNK (SEQ ID NO:20), SCICTVVQRCASNKP (SEQ ID NO:21), CICTVVQRCASNKPH (SEQ ID NO:22), ICTVVQRCASNKPHV (SEQ ID NO:23), CTVVQRCASNKPHVL (SEQ ID NO:24), TVVQRCASNKPHVLE (SEQ ID NO:25), VVQRCASNKPHVLED (SEQ ID NO:26), VQRCASNKPHVLEDP (SEQ ID NO:27), QRCASNKPHVLEDPC (SEQ ID NO:28), RCASNKPHVLEDPCK (SEQ ID NO:29), DPNQTNVVPPALHLV (SEQ ID NO:30), PNQTNVVPPALHLVD (SEQ ID NO:31), NQTNVVPPALHLVDP (SEQ ID NO:32), QTNVVPPALHLVDPQ (SEQ ID NO:33), TNVVPPALHLVDPQI (SEQ ID NO:34), NVVPPALHLVDPQIQ (SEQ ID NO:35), VVPPALHLVDPQIQL (SEQ ID NO:36), VPPALHLVDPQIQLT (SEQ ID NO:37), PPALHLVDPQIQLTI (SEQ ID NO:38), PALHLVDPQIQLTIT (SEQ ID NO:39), LTITRMEDAMGQGQN (SEQ ID NO:40), TITRMEDAMGQGQNS (SEQ ID NO:41), ITRMEDAMGQGQNSA (SEQ ID NO:42), TRMEDAMGQGQNSAD (SEQ ID NO:43), RMEDAMGQGQNSADP (SEQ ID NO:44), MEDAMGQGQNSADPK (SEQ ID NO:45), VVQMTKLATTEELPD (SEQ ID NO:46), VQMTKLATTEELPDE (SEQ ID NO:47), QMTKLATTEELPDEF (SEQ ID NO:48), MTKLATTEELPDEFV (SEQ ID NO:49), TKLATTEELPDEFVV (SEQ ID NO:50), KLATTEELPDEFVVV (SEQ ID NO:51), LATTEELPDEFVVVT (SEQ ID NO:52), ATTEELPDEFVVVTA (SEQ ID NO:53), LPPCYNFLKEQHCQK (SEQ ID NO:54), PPCYNFLKEQHCQKA (SEQ ID NO:55), PCYNFLKEQHCQKAS (SEQ ID NO:56), CYNFLKEQHCQKAST (SEQ ID NO:57), YNFLKEQHCQKASTQ (SEQ ID NO:58), NFLKEQHCQKASTQR (SEQ ID NO:59), FLKEQHCQKASTQRE (SEQ ID NO:60), LKEQHCQKASTQREA (SEQ ID NO:61), KEQHCQKASTQREAE (SEQ ID NO:62), EQHCQKASTQREAEA (SEQ ID NO:63), QHCQKASTQREAEAA (SEQ ID NO:64), HCQKASTQREAEAAV (SEQ ID NO:65), CQKASTQREAEAAVK (SEQ ID NO:66), QKASTQREAEAAVKP (SEQ ID NO:67), KASTQREAEAAVKPL (SEQ ID NO:68), ASTQREAEAAVKPLL (SEQ ID NO:69) and STQREAEAAVKPLLA (SEQ ID NO:70).

The peptides above are recognized in linear and/or looped/cyclic form by at least one of the following sera; a serum derived from an individual that has been infected by SARS-CoV and has recovered from SARS (serum called SARS-green), a serum derived from an individual in which the virus was still detectable by PCR and who suffered a prolonged and severe form of the illness (serum called SARS-yellow) and sera derived from individuals which have been and/or are infected by SARS-CoV (sera called 1a (individual 1, early serum), 1b (individual 1, late serum), 2 (individual 2), 6 (individual 6), 37 (individual 37), 62 (individual 62) and London).

In an embodiment of the invention, the invention encompasses a peptide having an amino acid sequence selected from the group consisting of LIMLIIFWFSLEIQD (SEQ ID NO:11), IMLIIFWFSLEIQDL (SEQ ID NO:12), MLIIFWFSLEIQDLE (SEQ ID NO:13), LIIFWFSLEIQDLEE (SEQ ID NO:14), IIFWFSLEIQDLEEP (SEQ ID NO:15), IFWFSLEIQDLEEPC (SEQ ID NO:16), FWFSLEIQDLEEPCT (SEQ ID NO:17), WFSLEIQDLEEPCTK (SEQ ID NO:18) and FSLEIQDLEEPCTKV (SEQ ID NO:19). These peptides are peptides of the (potential) protein Orf9 from SARS-CoV TOR2 and Orf7b from Frankfurt 1 and HSR 1. They are recognized in linear and in looped form.

The invention also encompasses a peptide having an amino acid sequence selected from the group consisting of CSCICTVVQRCASNK (SEQ ID NO:20), SCICTVVQRCASNKP (SEQ ID NO:21), CICTVVQRCASNKPH (SEQ ID NO:22), ICTVVQRCASNKPHV (SEQ ID NO:23), CTVVQRCASNKPHVL (SEQ ID NO:24), TVVQRCASNKPHVLE (SEQ ID NO:25), VVQRCASNKPHVLED (SEQ ID NO:26), VQRCASNKPHVLEDP (SEQ ID NO:27), QRCASNKPHVLEDPC (SEQ ID NO:28) and RCASNKPHVLEDPCK (SEQ ID NO:29). These peptides are peptides of protein Orf10 from SARS-CoV TOR2 and Orf8a from Frankfurt 1 and HSR 1. They are recognized in linear and in looped form.

The invention also encompasses a peptide having an amino acid sequence selected from the group consisting of DPNQTNVVPPALHLV (SEQ ID NO:30), PNQTNVVPPALHLVD (SEQ ID NO:31), NQTNVVPPALHLVDP (SEQ ID NO:32), QTNVVPPALHLVDPQ (SEQ ID NO:33), TNVVPPALHLVDPQI (SEQ ID NO:34), NVVPPALHLVDPQIQ (SEQ ID NO:35), VVPPALHLVDPQIQL (SEQ ID NO:36), VPPALHLVDPQIQLT (SEQ ID NO:37), PPALHLVDPQIQLTI (SEQ ID NO:38), PALHLVDPQIQLTIT (SEQ ID NO:39), LTITRMEDAMGQGQN (SEQ ID NO:40), TITRMEDAMGQGQNS (SEQ ID NO:41), ITRMEDAMGQGQNSA (SEQ ID NO:42), TRMEDAMGQGQNSAD (SEQ ID NO:43), RMEDAMGQGQNSADP (SEQ ID NO:44), MEDAMGQGQNSADPK (SEQ ID NO:45), VVQMTKLATTEELPD (SEQ ID NO:46), VQMTKLATTEELPDE (SEQ ID NO:47), QMTKLATTEELPDEF (SEQ ID NO:48), MTKLATTEELPDEFV (SEQ ID NO:49), TKLATTEELPDEFVV (SEQ ID NO:50), KLATTEELPDEFVVV (SEQ ID NO:51), LATTEELPDEFVVVT (SEQ ID NO:52) and ATTEELPDEFVVVTA (SEQ ID NO:53). These peptides are peptides of protein Orf13 from SARS-CoV TOR2 and Orf9b from Frankfurt 1 and HSR 1. The peptides having an amino acid sequence selected from the group consisting of DPNQTNVVPPALHLV (SEQ ID NO:30), PNQTNVVPPALHLVD (SEQ ID NO:31), NQTNVVPPALHLVDP (SEQ ID NO:32), QTNVVPPALHLVDPQ (SEQ ID NO:33), TNVVPPALHLVDPQI (SEQ ID NO:34), NVVPPALHLVDPQIQ (SEQ ID NO:35), VVPPALHLVDPQIQL (SEQ ID NO:36), VPPALHLVDPQIQLT (SEQ ID NO:37), PPALHLVDPQIQLTI (SEQ ID NO:38), PALHLVDPQIQLTIT (SEQ ID NO:39), VVQMTKLATTEELPD (SEQ ID NO:46), VQMTKLATTEELPDE (SEQ ID NO:47), QMTKLATTEELPDEF (SEQ ID NO:48), MTKLATTEELPDEFV (SEQ ID NO:49), TKLATTEELPDEFVV (SEQ ID NO:50), KLATTEELPDEFVVV (SEQ ID NO:51), LATTEELPDEFVVVT (SEQ ID NO:52) and ATTEELPDEFVVVTA (SEQ ID NO:53) are recognized in linear and in looped form. The peptides having an amino acid sequence selected from the group consisting of LTITRMEDAMGQGQN (SEQ ID NO:40), TITRMEDAMGQGQNS (SEQ ID NO:41), ITRMEDAMGQGQNSA (SEQ ID NO:42), TRMEDAMGQGQNSAD (SEQ ID NO:43), RMEDAMGQGQNSADP (SEQ ID NO:44), MEDAMGQGQNSADPK (SEQ ID NO:45) are only recognized in looped form.

In another embodiment, the invention encompasses a peptide having an amino acid sequence selected from the group consisting of LPPCYNFLKEQHCQK (SEQ ID NO:54), PPCYNFLKEQHCQKA (SEQ ID NO:55), PCYNFLKEQHCQKAS (SEQ ID NO:56), CYNFLKEQHCQKAST (SEQ ID NO:57), YNFLKEQHCQKASTQ (SEQ ID NO:58), NFLKEQHCQKASTQR (SEQ ID NO:59), FLKEQHCQKASTQRE (SEQ ID NO:60), LKEQHCQKASTQREA (SEQ ID NO:61), KEQHCQKASTQREAE (SEQ ID NO:62), EQHCQKASTQREAEA (SEQ ID NO:63), QHCQKASTQREAEAA (SEQ ID NO:64), HCQKASTQREAEAAV (SEQ ID NO:65), CQKASTQREAEAAVK (SEQ ID NO:66), QKASTQREAEAAVKP (SEQ ID NO:67), KASTQREAEAAVKPL (SEQ ID NO:68), ASTQREAEAAVKPLL (SEQ ID NO:69) and STQREAEAAVKPLLA (SEQ ID NO:70). These peptides are peptides of the protein Orf14 from SARS-CoV TOR2. They are recognized in looped form.

The oligopeptides identified above are good candidates to represent a neutralizing epitope of SARS-CoV, particularly the SARS-CoV strains TOR2, Frankfurt 1 and/or HSR 1. They may be used in therapy and/or prevention of conditions resulting from an infection with SARS-CoV as described herein and may also be used in diagnostic test methods as described herein.

In a further aspect of the invention, peptides mentioned above may be coupled/linked to each other. Peptides of the embodiments of the invention may be linked/coupled to peptides of other embodiments of the invention or the same embodiment of the invention. The peptides may be linear and/or looped/cyclic. A combination peptide may also constitute of more than two peptides. The peptides of the invention can be linked directly or indirectly via for instance a spacer of variable length. Furthermore, the peptides can be linked covalently or non-covalently. They may also be part of a fusion protein or conjugate.

A combination peptide which contains different peptides from one embodiment of the invention, i.e., form one protein, may mimic/simulate a discontinuous and/or conformational epitope. Such an epitope may be more antigenic than the single peptides. In general, the peptides should be in such a form as to be capable of mimicking/simulating a discontinuous and/or conformational epitope.

Obviously, the person skilled in the art may make modifications to the peptide without departing from the scope of the invention, e.g., by systematic length variation and/or replacement of residues and/or combination with other peptides. Peptides can be synthesized by known solid phase peptide synthesis techniques. The synthesis allows for one or more amino acids not corresponding to the original peptide sequence to be added to the amino or carboxyl terminus of the peptides. Such extra amino acids are useful for coupling the peptides to each other, to another peptide, to a large carrier protein or to a solid support. Amino acids that are inter alia useful for these purposes include tyrosine, lysine, glutamic acid, aspartic acid, cysteine and derivatives thereof. Additional protein modification techniques may be used, e.g., NH₂-acetylation or COOH-terminal amidation, to provide additional means for coupling the peptides to another protein or peptide molecule or to a support, for example, polystyrene or polyvinyl microtiter plates, glass tubes or glass beads or particles and chromatographic supports, such as paper, cellulose and cellulose derivates, and silica. If the peptide is coupled to such a support, it may also be used for affinity purification of SARS-CoV recognizing antibodies.

In an embodiment the peptides of the invention can have a looped/cyclic form. Linear peptides can be made by chemically converting the structures to looped/cyclic forms. It is well known in the art that cyclization of linear peptides can modulate bioactivity by increasing or decreasing the potency of binding to the target protein. Linear peptides are very flexible and tend to adopt many different conformations in solution. Cyclization acts to constrain the number of available conformations, and thus, favor the more active or inactive structures of the peptide. Cyclization of linear peptides is accomplished either by forming a peptide bond between the free N-terminal and C-terminal ends (homodetic cyclopeptides) or by forming a new covalent bond between amino acid backbone and/or side chain groups located near the N- or C-terminal ends (heterodetic cyclopeptides). The latter cyclizations use alternate chemical strategies to form covalent bonds, for example, disulfides, lactones, ethers, or thioethers. However, cyclization methods other than the ones described above can also be used to form cyclic/looped peptides. Generally, linear peptides of more than five residues can be cyclized relatively easily. The propensity of the peptide to form a beta-turn conformation in the central four residues facilitates the formation of both homo- and heterodetic cyclopeptides. The looped/cyclic peptides of the invention preferably comprise a cysteine residue at position 2 and 14. Preferably, they contain a linker between the cysteine residues. The looped/cyclic peptides of the invention are recognized by antibodies in the serum of individuals that have been and/or are infected with SARS-CoV.

Alternatively, the peptides of the invention may be prepared by expression of the peptides or of a larger peptide including the desired peptide from a corresponding gene (whether synthetic or natural in origin) in a suitable host. The larger peptide may contain a cleavage site whereby the peptide of interest may be released by cleavage of the fused molecule.

The resulting peptides may then be tested for binding to sera from subjects that have been previously infected with SARS-CoV, to sera from infected subjects or to purified SARS-CoV antibodies in a way essentially as described herein. If such a peptide can still be bound by the sera or antibody, it is considered as a functional fragment or analogue of the peptides according to the invention. Also, even stronger antigenic peptides may be identified in this manner, which peptides may be used for vaccination purposes or for generating strongly neutralizing antibodies for therapeutic or prophylactic purposes. The peptides may also be used in diagnostic tests. Therefore the invention also provides the peptides comprising a part (or even consisting of a part) of a peptide according to the invention, wherein the part is recognized by antibodies present in serum derived from an individual that has been and/or is infected by SARS-CoV.

Furthermore, the invention provides peptides consisting of an analogue of a peptide according to the invention, wherein one or more amino acids are substituted for another amino acid, and wherein the analogue is recognized by antibodies present in serum derived from an individual that has been and/or is infected by SARS-CoV. Alternatively, analogues can be peptides of the present invention comprising an amino acid sequence containing insertions, deletions or combinations thereof of one or more amino acids compared to the amino acid sequences of the parent peptides. Furthermore, analogues can comprise truncations of the amino acid sequence at either or both the amino or carboxy termini of the peptides. Analogues according to the invention may have the same or different, either higher or lower, antigenic properties compared to the parent peptides, but are still recognized by antibodies present in serum derived from an individual that has been and/or is infected by SARS-CoV. That part of a 15-mer still representing immunogenic activity consists of about six to twelve, preferably eight to ten, more preferably, nine amino acids within the 15-mer.

The peptides, parts thereof or analogues thereof according to the invention may be used directly as peptides, but may also be used conjugated to an immunogenic carrier, which may be, e.g., a polypeptide or polysaccharide. If the carrier is a polypeptide, the desired conjugate may be expressed as a fusion protein. Alternatively, the peptide and the carrier may be obtained separately and then conjugated. This conjugation may be covalently or non-covalently. A fusion protein is a chimeric protein, comprising the peptide according to the invention, and another protein or part thereof not being a SARS-CoV protein. Such fusion proteins may for instance be used to raise antibodies for diagnostic, prophylactic or therapeutic purposes or to directly immunize, i.e., vaccinate, humans or animals. Any protein or part thereof or even peptide may be used as fusion partner for the peptide according to the invention to form a fusion protein, and non-limiting examples are bovine serum albumin, keyhole limpet hemocyanin, etc.

The peptides may be labeled (signal-generating) or unlabeled. This depends on the type of assay used. Labels which may be coupled to the peptides are those known in the art and include, but are not limited to, enzymes, radionuclides, fluorogenic and chromogenic substrates, cofactors, biotin/avidin, colloidal gold, and magnetic particles.

It is another aspect of the invention to provide nucleic acid molecules encoding peptides, parts thereof or analogues thereof or fusion proteins according to the invention. Such nucleic acid molecules may suitably be used in the form of plasmids for propagation and expansion in bacterial or other hosts. Moreover, recombinant DNA techniques well known to the person skilled in the art can be used to obtain nucleic acid molecules encoding analogues of the peptides according to the invention, e.g., by mutagenesis of the sequences encoding the peptides according to the invention. The skilled man will appreciate that analogues of the nucleic acid molecules are also intended to be a part of the present invention. Analogues are also nucleic acid sequences that can be directly translated, using the standard genetic code, to provide an amino acid sequence identical to that translated from the parent nucleic acid molecules. Another aspect of nucleic acid molecules according to the present invention, is their potential for use in gene-therapy or vaccination applications. Therefore, in another embodiment of the invention, nucleic acid molecules according to the invention are provided wherein the nucleic acid molecule is present in a gene delivery vehicle. A “gene delivery vehicle” as used herein refers to an entity that can be used to introduce nucleic acid molecules into cells, and includes liposomes, naked DNA, plasmid DNA, optionally coupled to a targeting moiety such as an antibody with specificity for an antigen presenting cell, recombinant viruses, and the like. Preferred gene therapy vehicles of the present invention will generally be viral vectors, such as comprised within a recombinant retrovirus, herpes simplex virus (HSV), adenovirus, adeno-associated virus (AAV), cytomegalovirus (CMV), and the like. Such applications of the nucleic acid sequences according to the invention are included in the present invention. The person skilled in the art will be aware of the possibilities of recombinant viruses for administering sequences of interest to cells. The administration of the nucleic acids of the invention to cells can result in an enhanced immune response. Alternatively, the nucleic acid encoding the peptides of the invention can be used as naked DNA vaccines, e.g., immunization by injection of purified nucleic acid molecules into humans or animals.

In another aspect, the invention provides antibodies recognizing the peptides, parts or analogues thereof of the invention. Antibodies can be obtained according to routine methods well known to the person skilled in the art, including but not limited to immunization of animals such as mice, rabbits, goats, and the like, or by antibody, phage or ribosome display methods (see e.g., Using Antibodies: A Laboratory Manual, Edited by: E. Harlow, D. Lane (1998), Cold Spring Harbor Laboratory Press, Cold Spring Harbor, N.Y.; Current Protocols in Immunology, Edited by: J. E. Coligan, A. M. Kruisbeek, D. H. Margulies, E. M. Shevach, W. Strober (2001), John Wiley & Sons Inc., New York; and Phage Display: A Laboratory Manual. Edited by: C. F. Barbas, D. R. Burton, J. K. Scott and G. J. Silverman (2001), Cold Spring Harbor Laboratory Press, Cold Spring Harbor, N.Y., the disclosures of which are incorporated herein by reference).

The antibodies of the invention can be intact immunoglobulin molecules such as polyclonal or monoclonal antibodies, in particular human monoclonal antibodies, or the antibodies can be functional fragments thereof, i.e., fragments that are still capable of binding to the antigen. These fragments include, but not limited to, Fab, F(ab′), F(ab′)₂, Fv, dAb, Fd, complementarity determining region (CDR) fragments, single-chain antibodies (scFv), bivalent single-chain antibodies, diabodies, triabodies, tetrabodies, and (poly)peptides that contain at least a fragment of an immunoglobulin that is sufficient to confer specific antigen binding to the (poly)peptides. The antibodies of the invention can be used in non-isolated or isolated form. Furthermore, the antibodies of the invention can be used alone or in a mixture/composition comprising at least one antibody (or variant or fragment thereof) of the invention. Antibodies of the invention include all the immunoglobulin classes and subclasses known in the art. Depending on the amino acid sequence of the constant domain of their heavy chains, binding molecules can be divided into the five major classes of intact antibodies: IgA, IgD, IgE, IgG, and IgM, and several of these may be further divided into subclasses (isotypes), e.g., IgA1, IgA2, IgG1, IgG2, IgG3 and IgG4. The above mentioned antigen-binding fragments may be produced synthetically or by enzymatic or chemical cleavage of intact immunoglobulins or they may be genetically engineered by recombinant DNA techniques. The methods of production are well known in the art and are described, for example, in Antibodies: A Laboratory Manual, Edited by: E. Harlow and D, Lane (1988), Cold Spring Harbor Laboratory Press, Cold Spring Harbor, N.Y., which is incorporated herein by reference. A binding molecule or antigen-binding fragment thereof may have one or more binding sites. If there is more than one binding site, the binding sites may be identical to one another or they may be different.

The antibodies of the invention can be naked or unconjugated antibodies. A naked or unconjugated antibody is intended to refer to an antibody that is not conjugated, operatively linked or otherwise physically or functionally associated with an effector moiety or tag, such as inter alia a toxic substance, a radioactive substance, a liposome, an enzyme. It will be understood that naked or unconjugated antibodies do not exclude antibodies that have been stabilized, multimerized, humanized or in any other way manipulated, other than by the attachment of an effector moiety or tag. Accordingly, all post-translationally modified naked and unconjugated antibodies are included herewith, including where the modifications are made in the natural antibody-producing cell environment, by a recombinant antibody-producing cell, and are introduced by the hand of man after initial antibody preparation. Of course, the term naked or unconjugated antibody does not exclude the ability of the antibody to form functional associations with effector cells and/or molecules after administration to the body, as some of such interactions are necessary in order to exert a biological effect. The lack of associated effector group or tag is therefore applied in definition to the naked or unconjugated binding molecule in vitro, not in vivo.

Alternatively, the antibodies as described in the present invention can be conjugated to tags and be used for detection and/or analytical and/or diagnostic purposes. The tags used to label the antibodies for those purposes depend on the specific detection/analysis/diagnosis techniques and/or methods used such as inter alia immunohistochemical staining of tissue samples, flow cytometric detection, scanning laser cytometric detection, fluorescent immunoassays, enzyme-linked immunosorbent assays (ELISAs), radioimmunoassays (RIAs), bioassays (e.g., neutralization assays, growth inhibition assays), Western blotting applications, etc. For immunohistochemical staining of tissue samples preferred labels are enzymes that catalyze production and local deposition of a detectable product. Enzymes typically conjugated to antibodies to permit their immunohistochemical visualization are well-known and include, but are not limited to, alkaline phosphatase, P-galactosidase, glucose oxidase, horseradish peroxidase, and urease. Typical substrates for production and deposition of visually detectable products include, but are not limited to, o-nitrophenyl-beta-D-galactopyranoside (ONPG), o-phenylenediamine dihydrochloride (OPD), p-nitrophenyl phosphate (PNPP), p-nitrophenyl-beta-D-galactopryanoside (PNPG), 3′,3′-diaminobenzidine (DAB), 3-amino-9-ethylcarbazole (AEC), 4-chloro-1-naphthol (CN), 5-bromo-4-chloro-3-indolyl-phosphate (BCIP), ABTS, BluoGal, iodonitrotetrazolium (INT), nitroblue tetrazolium chloride (NBT), phenazine methosulfate (PMS), phenolphthalein monophosphate (PMP), tetramethyl benzidine (TMB), tetranitroblue tetrazolium (TNBT), X-Gal, X-Gluc, and X-glucoside. Other substrates that can be used to produce products for local deposition are luminescent substrates. For example, in the presence of hydrogen peroxide, horseradish peroxidase can catalyze the oxidation of cyclic diacylhydrazides such as luminol. Next to that, binding molecules of the immunoconjugate of the invention can also be labeled using colloidal gold or they can be labeled with radioisotopes, such as ³³p, ³²p, ³⁵S, ³H, and ¹²⁵I. When the antibodies of the present invention are used for flow cytometric detections, scanning laser cytometric detections, or fluorescent immunoassays, they can usefully be labeled with fluorophores. A wide variety of fluorophores useful for fluorescently labeling the antibodies of the present invention include, but are not limited to, Alexa Fluor and Alexa Fluor&commat dyes, BODIPY dyes, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethylrhodamine, Cy2, Cy3, Cy3.5, Cy5, Cy5.5, Cy7, fluorescein isothiocyanate (FITC), allophycocyanin (APC), R-phycoerythrin (PE), peridinin chlorophyll protein (PerCP), Texas Red, fluorescence resonance energy tandem fluorophores such as PerCP-Cy5.5, PE-Cy5, PE-Cy5.5, PE-Cy7, PE-Texas Red, and APC-Cy7. When the antibodies of the present invention are used for secondary detection using labeled avidin, streptavidin, captavidin or neutravidin, the antibodies may be labeled with biotin.

Next to that, the antibodies of the invention may be conjugated to photoactive agents or dyes such as fluorescent and other chromogens or dyes to use the so obtained immunoconjugates in photoradiation, phototherapy, or photodynamic therapy. The photoactive agents or dyes include, but are not limited to, photofrin®, synthetic diporphyrins and dichlorins, phthalocyanines with or without metal substituents, chloroaluminum phthalocyanine with or without varying substituents, O-substituted tetraphenyl porphyrins, 3,1-meso tetrakis (O-propionamido phenyl) porphyrin, verdins, purpurins, tin and zinc derivatives of octaethylpurpurin, etiopurpurin, hydroporphyrins, bacteriochlorins of the tetra(hydroxyphenyl) porphyrin series, chlorins, chlorin e₆, mono-1-aspartyl derivative of chlorin e₆, di-1-aspartyl derivative of chlorin e₆, tin(IV) chlorin e₆, meta-tetrahydroxyphenylchlor-in, benzoporphyrin derivatives, benzoporphyrin monoacid derivatives, tetracyanoethylene adducts of benzoporphyrin, dimethyl acetylenedicarboxylate adducts of benzoporphyrin, Diels-Adler adducts, monoacid ring “a” derivative of benzoporphyrin, sulfonated aluminum PC, sulfonated AlPc, disulfonated, tetrasulfonated derivative, sulfonated aluminum naphthalocyanines, naphthalocyanines with or without metal substituents and with or without varying substituents, anthracenediones, anthrapyrazoles, aminoanthraquinone, phenoxazine dyes, phenothiazine derivatives, chalcogenapyrylium dyes, cationic selena and tellurapyrylium derivatives, ring-substituted cationic PC, pheophorbide derivative, naturally occurring porphyrins, hematoporphyrin, ALA-induced protoporphyrin IX, endogenous metabolic precursors, 5-aminolevulinic acid benzonaphthoporphyrazines, cationic imminium salts, tetracyclines, lutetium texaphyrin, tin-etio-purpurin, porphycenes, benzophenothiazinium and combinations thereof.

When the antibodies of the invention are used for in vivo diagnostic use, the antibodies can also be made detectable by conjugation to e.g., magnetic resonance imaging (MRI) contrast agents, such as gadolinium diethylenetriaminepentaacetic acid, to ultrasound contrast agents or to X-ray contrast agents, or by radioisotopic labeling.

The antibodies according to the invention may be capable of neutralizing SARS-CoV infectivity and are useful for therapeutic purposes against this virus. Assays to detect and measure virus neutralizing activity of antibodies are well known in the art. For example, a SARS-CoV neutralization assay can be performed on Vero cells (ATCC CCL 81). Antibodies of the invention are mixed with virus suspension and incubated for one hour at 37° C. The obtained suspension is then inoculated onto sub-confluent Vero cells (approximately 80% density) grown in 96-well cell culture plates. The inoculated cells are cultured for three to four days at 37° C. and observed daily for the development of cytopathic effect (CPE). CPE is compared to the positive control (virus inoculated cells) and negative controls (mock-inoculated cells or cells incubated with antibody only). Alternatively, the antibodies may inhibit or down-regulate SARS-CoV replication, are complement fixing antibodies capable of assisting in the lysis of enveloped SARS-CoV and/or act as opsonins and augment phagocytosis of SARS-CoV either by promoting its uptake via Fc or C3b receptors or by agglutinating SARS-CoV to make it more easily phagocytosed.

The invention also provides nucleic acid molecules encoding the antibodies according to the invention.

It is another aspect of the invention to provide vectors, i.e., nucleic acid constructs, comprising one or more nucleic acid molecules according to the present invention. The nucleic acid molecule may either encode the peptides, parts or analogues thereof or fusion proteins of the invention or encode the antibodies of the invention. Vectors can be derived from plasmids such as inter alia F, R1, RP1, Col, pBR322, TOL, Ti, etc; cosmids; phages such as lambda, lambdoid, M13, Mu, P1, P22, Q_{β}, T-even, T-odd, T2, T4, T7, etc; plant viruses such as inter alia alfalfa mosaic virus, bromovirus, capillovirus, carlavirus, carmovirus, caulivirus, clostervirus, comovirus, cryptovirus, cucumovirus, dianthovirus, fabavirus, fijivirus, furovirus, geminivirus, hordeivirus, ilarvirus, luteovirus, machlovirus, marafivirus, necrovirus, nepovirus, phytorepvirus, plant rhabdovirus, potexvirus, potyvirus, sobemovirus, tenuivirus, tobamovirus, tobravirus, tomato spotted wilt virus, tombusvirus, tymovirus, etc; or animal viruses such as inter alia adenovirus, arenaviridae, baculoviridae, bimaviridae, bunyaviridae, calciviridae, cardioviruses, coronaviridae, corticoviridae, cystoviridae, Epstein-Barr virus, enteroviruses, filoviridae, flaviviridae, Foot-and-Mouth disease virus, hepadnaviridae, hepatitis viruses, herpesviridae, immunodeficiency viruses, influenza virus, inoviridae, iridoviridae, orthomyxoviridae, papovaviruses, paramyxoviridae, parvoviridae, picornaviridae, poliovirus, polydnaviridae, poxyiridae, reoviridae, retroviruses, rhabdoviridae, rhinoviruses, Semliki Forest virus, tetraviridae, togaviridae, toroviridae, vaccinia virus, vescular stomatitis virus, etc. Vectors can be used for cloning and/or for expression of the peptides, parts or analogues thereof of the invention or antibodies of the invention of the invention and might even be used for gene therapy purposes. Vectors comprising one or more nucleic acid molecules according to the invention operably linked to one or more expression-regulating nucleic acid molecules are also covered by the present invention. The choice of vector is dependent on the recombinant procedures followed and the host used. Introduction of vectors in host cells can be effected by inter alia calcium phosphate transfection, virus infection, DEAE-dextran mediated transfection, lipofectamin transfection or electroporation. Vectors may be autonomously replicating or may replicate together with the chromosome into which they have been integrated. Preferably, the vectors contain one or more selection markers. Useful markers are dependent on the host cells of choice and are well known to persons skilled in the art. They include, but are not limited to, kanamycin, neomycin, puromycin, hygromycin, zeocin, thymidine kinase gene from Herpes simplex virus (HSV-TK), dihydrofolate reductase gene from mouse (dhfr). Vectors comprising one or more nucleic acid molecules encoding the peptides, parts or analogues thereof or antibodies as described above operably linked to one or more nucleic acid molecules encoding proteins or peptides that can be used to isolate these molecules are also covered by the invention. These proteins or peptides include, but are not limited to, glutathione-S-transferase, maltose binding protein, metal-binding polyhistidine, green fluorescent protein, luciferase and beta-galactosidase.

Hosts containing one or more copies of the vectors mentioned above are an additional subject of the present invention. Preferably, the hosts are cells. Preferably, the cells are suitably used for the manipulation and propagation of nucleic acid molecules. Suitable cells include, but are not limited to, cells of mammalian, plant, insect, fungal or bacterial origin. Bacterial cells include, but are not limited to, cells from Gram positive bacteria such as several species of the genera Bacillus, Streptomyces and Staphylococcus or cells of Gram negative bacteria such as several species of the genera Escherichia, such as Escherichia coli, and Pseudomonas. In the group of fungal cells preferably yeast cells are used. Expression in yeast can be achieved by using yeast strains such as inter alia Pichia pastoris, Saccharomyces cerevisiae and Hansenula polymorpha. Furthermore, insect cells such as cells from Drosophila and Sf9 can be used as host cells. Besides that, the host cells can be plant cells such as inter alia cells from crop plants such as forestry plants, or cells from plants providing food and raw materials such as cereal plants, or medicinal plants, or cells from ornamentals, or cells from flower bulb crops. Transformed (transgenic) plants or plant cells are produced by known methods, for example, Agrobacterium-mediated gene transfer, transformation of leaf discs, protoplast transformation by polyethylene glycol-induced DNA transfer, electroporation, sonication, microinjection or bolistic gene transfer. Additionally, a suitable expression system can be a baculovirus system. Expression systems using mammalian cells such as Chinese Hamster Ovary (CHO) cells, COS cells, BHK cells or Bowes melanoma cells are preferred in the present invention. Mammalian cells provide expressed proteins with posttranslational modifications that are most similar to natural molecules of mammalian origin. Since the present invention deals with molecules that may have to be administered to humans, a completely human expression system would be particularly preferred. Therefore, even more preferably, the host cells are human cells. Examples of human cells are inter alia HeLa, 911, AT1080, A549, 293 and HEK293T cells. Preferred mammalian cells are human retina cells such as 911 cells or the cell line deposited at the European Collection of Cell Cultures (ECACC), CAMR, Salisbury, Wiltshire SP4 OJG, Great Britain on 29 Feb. 1996 under number 96022940 and marketed under the trademark PER.C6® (PER.C6 is a registered trademark of Crucell Holland B.V.). For the purposes of this application “PER.C6” refers to cells deposited under number 96022940 or ancestors, passages up-stream or downstream as well as descendants from ancestors of deposited cells, as well as derivatives of any of the foregoing. The cells according to the invention may contain the nucleic acid molecule according to the invention in expressible format, such that the desired protein can be recombinantly expressed from the cells.

In a further aspect, the invention is directed to a peptide, part or analogue thereof according to the invention, or a fusion protein according to the invention or a nucleic acid molecule encoding a peptide, part or analogue thereof according to the invention or a nucleic acid molecule encoding a fusion protein of the invention for use as a medicament. In other words, the invention is directed to a method of prevention and/or treatment wherein a peptide, part or analogue thereof according to the invention, or a fusion protein according to the invention or a nucleic acid molecule encoding a peptide, part or analogue thereof according to the invention or a nucleic acid molecule encoding a fusion protein of the invention is used. Preferably, the peptides, parts or analogues thereof of the invention may for example be for use as an immunogen, preferably a vaccine.

If the peptides, parts and analogues thereof of the invention are in the form of a vaccine, they are preferably formulated into compositions. A composition may also comprise more than one peptide of the invention. These peptides may be different or identical and may be linked, covalently or non-covalently, to each other or not linked to each other. They may be linear and/or looped/cyclic. For formulation of such compositions, an immunogenically effective amount of at least one of the peptides of the invention is admixed with a physiologically acceptable carrier suitable for administration to animals including man. The peptides may be covalently attached to each other, to other peptides, to a protein carrier or to other carriers, incorporated into liposomes or other such vesicles, or complexed with an adjuvant or adsorbent as is known in the vaccine art. Alternatively, the peptides are not complexed with the any of the above molecules and are merely admixed with a physiologically acceptable carrier such as normal saline or a buffering compound suitable for administration to animals including man. As with all immunogenic compositions for eliciting antibodies, the immunogenically effective amounts of the peptides of the invention must be determined. Factors to be considered include the immunogenicity of the native peptide, whether or not the peptide will be complexed with or covalently attached to an adjuvant or carrier protein or other carrier and route of administration for the composition, i.e., intravenous, intramuscular, subcutaneous, etc., and number of immunizing doses to be administered. Such factors are known in the vaccine art and it is well within the reach of a skilled artisan to make such determinations without undue experimentation. The peptides, parts or analogues thereof or compositions comprising these compounds may elicit an antibody response upon administrating to human or animal subjects. Such an antibody response protects against further infection by SARS-CoV and/or will retard the onset or progress of the symptoms associated with SARS.

Most preferably, they can be used in the treatment of a condition resulting from a SARS-CoV, particularly from the SARS-CoV strains TOR2, Frankfurt 1 and HSR 1, as the peptides of the invention are derived form (potential) proteins of the SARS-CoV strains TOR2, Frankfurt 1 and HSR 1.

In yet another aspect, antibodies of the invention can be used as a medicament, preferably in the treatment of a condition resulting from a SARS-CoV such as the SARS-CoV strains TOR2, Frankfurt 1 and HSR 1. In a specific embodiment, they can be used with any other medicament available to treat a condition resulting from a SARS-CoV. In other words, the invention also pertains to a method of prevention and/or treatment, wherein the antibodies, fragments or functional variants thereof according to the invention are used.

The antibodies of the invention can also be used for detection of the SARS-CoV, e.g., for diagnostic purposes. Therefore, the invention provides a diagnostic test method for determining the presence of SARS-CoV in a sample, characterized in that the sample is put into contact with an antibody according to the invention. Preferably the antibody is contacted with the sample under conditions which allow the formation of an immunological complex between the antibodies and SARS-CoV or fragments or (poly)peptides thereof that may be present in the sample. The formation of an immunological complex, if any, indicating the presence of SARS-CoV in the sample, is then detected and measured by suitable means. The sample may be a biological sample including, but not limited to blood, serum, urine, tissue or other biological material from (potentially) infected subjects, or a nonbiological sample such as water, drink, etc. The (potentially) infected subjects may be human subjects, but also animals that are suspected as carriers of SARS-CoV might be tested for the presence of SARS-CoV using these antibodies. Detection of binding may be according to standard techniques known to a person skilled in the art, such as an ELISA, Western blot, RIA, etc. The antibodies may suitably be included in kits for diagnostic purposes. It is, therefore, another aspect of the invention to provide a kit of parts for the detection of SARS-CoV comprising an antibody according to the invention.

The antibodies of the invention may be used to purify SARS-CoV or a fragment thereof. Antibodies against peptides of specific proteins of SARS-CoV such as the proteins mentioned herein, may also be used to purify the proteins. Purification techniques for viruses and proteins are well known to the skilled artisan.

Also the peptide of the invention can be used directly for the detection of SARS-CoV recognizing antibodies, for instance for diagnostic purposes. It is therefore an object of the invention to provide methods for determining the presence of antibodies recognizing SARS-CoV in a sample, characterized in that the sample is put into contact with a peptide of the invention. Preferably the peptide is contacted with the sample under conditions which allow the formation of an immunological complex between the peptide and any antibodies to SARS-CoV that may be present in the sample. The formation of an immunological complex, if any, indicating the presence of antibodies to SARS-CoV in the sample, is then detected and measured by suitable means. Such methods include, inter alia, homogeneous and heterogeneous binding immunoassays, such as radioimmunoassays (RIA), ELISA and Western blot analyses. Further, the assay protocols using the novel peptides allow for competitive and non-competitive binding studies to be performed. The sample used in the diagnostic test method may for instance be blood, urine, tissue material or other material from (potentially) infected subjects. The peptide may however also be used to diagnose prior exposure to the SARS-CoV. Preferred assay techniques, especially for large-scale clinical screening of patient sera and blood and blood-derived products are ELISA and Western blot techniques. ELISA tests are particularly preferred. For use as reagents in these assays, the peptides of the invention are conveniently bonded to the inside surface of microtiter wells. The peptides may be directly bonded to the microtiter well. However, maximum binding of the peptides to the wells might be accomplished by pretreating the wells with polylysine prior to the addition of the peptides. Furthermore, the novel peptides may be covalently attached by known means to a carrier protein, such as BSA, with the resulting conjugate being used to coat the wells. Generally the peptides are used in a concentration of between 0.01 to 100 μg/ml for coating, although higher as well as lower amounts may also be used. Samples are then added to the peptide coated wells where an immunological complex forms if antibodies to SARS-CoV are present in the sample. A signal generating means may be added to aid detection of complex formation. A detectable signal is produced if SARS-CoV specific antibodies are present in the sample.

The peptides and antibodies of the invention can advantageously be used for detecting specific strains of SARS-CoV. As the peptides of the invention are part of (potential) proteins of the SARS-CoV strains TOR2, Frankfurt 1 and HSR 1, the peptides are suitable for recognizing antibodies specific for these strains in sera of individuals that have been and/or are infected with SARS-CoV. The antibodies of the invention can be used to detect the specific SARS-CoV strains TOR2, Frankfurt 1 and HSR 1 in sera of individuals that have been and/or are infected with SARS-CoV. The antibodies of the invention might also be used for strain specific purification purposes.

In a further aspect, the invention provides an isolated polypeptide comprising at least one of the peptides of the invention. Preferably, the isolated polypeptide comprises an amino acid sequence selected from the group consisting of SEQ ID NO:1, SEQ ID NO:2, SEQ ID NO:3, SEQ ID NO:4, SEQ ID NO:5, SEQ ID NO:6, SEQ ID NO:7, SEQ ID NO:8, SEQ ID NO:9 and SEQ ID NO:10. These polypeptides are potential proteins of the SARS-CoV strains TOR2, HSR 1 or Frankfurt 1. Binding of sera form SARS patients which have been and/or are infected with SARS-CoV to peptides of these proteins is the first indication that these proteins actually exist in nature.

### EXAMPLES

PEPSCAN-ELISA

Overlapping 15-mer linear and looped/cyclic peptides were synthesized from several (potential) proteins of SARS-CoV TOR2, Frankfurt 1 and HSR 1. The complete genome of these SARS-CoV strains can be found under EMBL-database accession numbers AY274119, AY291315 and AY323977, respectively. The coding sequence (CDS) of the (potential) proteins can also be found under the EMBL-database accession numbers indicated above.

Linear as well as looped/cyclic peptides were prepared from the SARS-CoV TOR2 (potential) proteins called protein Orf9 (the protein-id of protein Orf9 is AAP41044, see also SEQ ID NO:1), protein Orf10 (the protein-id of Orf10 is AAP41045, see also SEQ ID NO:2), protein Orf13 (the protein-id of protein Orf13 is AAP41048, see also SEQ ID NO:3), and Orf14 (the protein-id of Orf14 is AAP41049, see also SEQ ID NO:4). The protein Orf9 of the strain TOR2 is identical to the (potential) protein called Orf7b of strain Frankfurt 1 (the protein-id of Orf7b is AAP33704, see also SEQ ID NO:5) and to the (potential) protein Orf7b of strain HSR 1 (the protein-id of Orf7b is AAP72981, see also SEQ ID NO:6). The protein Orf10 of the strain TOR2 is identical to the potential protein called Orf8a of strain Frankfurt 1 (the protein-id of Orf8a is AAP33705, see also SEQ ID NO:9) and to the (potential) protein called Orf8a of strain HSR 1 (the protein-id of Orf8a is AAP72982, see also SEQ ID NO:10). The protein Orf13 of the strain TOR2 is identical to the (potential) protein called Orf9b of strain Frankfurt 1 (the protein-id of Orf9b is AAP33708, see also SEQ ID NO:7) and to the (potential) protein called Orf9b of strain HSR 1 (the protein-id of Orf9b is AAP72985, see also SEQ ID NO:8).

Next, the prepared peptides were screened using credit-card format mini-PEPSCAN cards (455 peptide formats/card) as described previously (Slootstra et al., 1996; WO 93/09872). All peptides were acetylated at the amino terminus.

In all looped peptides position-2 and position-14 were replaced by a cysteine (acetyl-XCXXXXXXXXXXXCX-minicard). If other cysteines besides the cysteines at position-2 and position-14 were present in a prepared peptide, the other cysteines were replaced by an alanine. The looped peptides were synthesized using standard Fmoc-chemistry and deprotected using trifluoric acid with scavengers. Subsequently, the deprotected peptides were reacted on the cards with an 0.5 mM solution of 1,3-bis(bromomethyl)benzene in ammonium bicarbonate (20 mM, pH 7.9)/acetonitril (1:1 (v/v)). The cards were gently shaken in the solution for 30 to 60 minutes, while completely covered in the solution. Finally, the cards were washed extensively with excess of H₂O and sonicated in disrupt-buffer containing 1% SDS/0.1% beta-mercaptoethanol in PBS (pH 7.2) at 70° C. for 30 minutes, followed by sonication in H₂O for another 45 minutes.

The binding of antibodies to each linear and looped peptide was tested in a PEPSCAN-based enzyme-linked immuno assay (ELISA). The 455-well creditcard-format polypropylene cards, containing the covalently linked peptides, were incubated with serum (diluted 1/1000 in blocking solution which contains 5% horse-serum (v/v) and 5% ovalbumin (w/v)) (4° C., overnight). Before use, the serum was heat-inactivated at 56° C. for one hour. After washing the peptides were incubated with anti-human antibody peroxidase (dilution 1/1000) (one hour, 25° C.), and subsequently, after washing the peroxidase substrate 2,2′-azino-di-3-ethylbenzthiazoline sulfonate (ABTS) and 2 μl/ml 3% H₂O₂ were added. After one hour, the color development was measured. The color development of the ELISA was quantified with a CCD-camera and an image processing system. The setup consists of a CCD-camera and a 55 mm lens (Sony CCD Video Camera XC-77RR, Nikon micro-nikkor 55 mm f/2.8 lens), a camera adaptor (Sony Camera adaptor DC-77RR) and the Image Processing Software package Optimas, version 6.5 (Media Cybernetics, Silver Spring, Md. 20910, U.S.A.). Optimas runs on a pentium II computer system.

Results

The serum derived from an individual that has been infected by SARS-CoV and has recovered from SARS (serum called SARS-green) and the serum derived from an individual in which the virus was still detectable by PCR and who suffered a prolonged and severe form of the illness (serum called SARS-yellow) and the sera derived from individuals which have been recovered from SARS-CoV and/or are still infected by SARS-CoV (the sera called 1a (individual 1, early serum), 1b (individual 1, late serum), 2 (individual 2), 6 (individual 6), 37 (individual 37), 62 (individual 62) and London) were tested for binding to the 15-mer linear and looped/cyclic peptides synthesized as described supra.

Additionally, two control sera were tested for binding the 15-mer linear and looped/cyclic peptides synthesized as described supra. One control serum was a pooled serum of ten healthy LUMC (Leids Universitair Medisch Centrum) hospital workers and the second control serum was a commercial negative donor pooled serum from the Dutch bloodbank. Next to that, a rabbit serum obtained by immunizing a rabbit with the SARS-CoV strain Frankfurt 1 was tested for binding the 15-mer linear and looped/cyclic peptides synthesized as described supra. The SARS-CoV was concentrated and partially purified by sucrose-gradient ultracentrifugation. After that, the purified SARS-CoV was gamma-irradiated for inactivation (approximately 35 kGy), mixed with complete Freund adjuvans and used for immunization purposes. Immunization was performed according to the art well known to the skilled artisan.

See Table 1 for results of the binding of the different above sera to linear peptides of Orf9 of SARS-CoV TOR2. See Table 2 for results of the binding of the different above sera to looped/cyclic peptides of Orf9 of SARS-CoV TOR2.

See Table 3 for results of the binding of the different above sera to linear peptides of Orf10 of SARS-CoV TOR2. See Table 4 for results of the binding of the different above sera to looped/cyclic peptides of Orf10 of SARS-CoV TOR2.

See Table 5 for results of the binding of the different above sera to linear peptides of Orf13 of SARS-CoV TOR2. See Table 6 for results of the binding of the different above sera to looped/cyclic peptides of Orf13 of SARS-CoV TOR2.

See Table 7 for results of the binding of the different above sera to linear peptides of Orf14 of SARS-CoV TOR2. See Table 8 for results of the binding of the different above sera to looped/cyclic peptides of Orf14 of SARS-CoV TOR2.

See Table 9 for results of the binding of the two control sera to linear and looped/cyclic peptides of Orf9 of SARS-CoV TOR2.

See Table 10 for results of the binding of the two control sera to linear and looped/cyclic peptides of Orf10 of SARS-CoV TOR2. The following peptides were recognized by at least one of the control sera in linear form, looped/cyclic form or in both forms: ICTVVQRCASNKPHV, CTVVQRCASNKPHVL and TVVQRCASNKPHVLE.

See Table 11 for results of the binding of the two control sera to linear and looped/cyclic peptides of Orf13 of SARS-CoV TOR2. The following peptides were recognized by at least one of the control sera in linear form, looped/cyclic form or in both forms: TNVVPPALHLVDPQI, NVVPPALHLVDPQIQ, VVPPALHLVDPQIQL and VPPALHLVDPQIQLT.

See Table 12 for results of the binding of the two control sera to linear and looped/cyclic peptides of Orf14 of SARS-CoV TOR2.

In Table 13 the results of the binding of the rabbit serum to linear and looped/cyclic peptides of Orf9 of SARS-CoV TOR2 are shown.

In Table 14 the results of the binding of the rabbit serum to linear and looped/cyclic peptides of Orf10 of SARS-CoV TOR2 are shown.

In Table 15 the results of the binding of the rabbit serum to linear and looped/cyclic peptides of Orf13 of SARS-CoV TOR2 are shown.

In Table 16 the results of the binding of the rabbit serum to linear and looped/cyclic peptides of Orf14 of SARS-CoV TOR2 are shown. The following peptides were recognized by the rabbit serum in linear form, looped/cyclic form or in both forms: EAAVKPLLAPHHVVA, AAVKPLLAPHHVVAV, AVKPLLAPHHVVAVI and VKPLLAPHHVVAVIQ.

The oligopeptides identified by the rabbit serum might be (additional) good candidates to represent epitopes of the SARS-CoV. The peptides may be advantageously used in diagnostic test methods as described herein. They may also be used in therapy and/or prevention of conditions resulting from an infection with SARS-CoV as described herein.

Relevant binding of a peptide to a serum was calculated as follows. The average OD-value for each serum was calculated for each protein (sum of OD-values of all peptides/total number of peptides). Next, the standard deviation of this average was calculated. The standard deviation was multiplied by two and the obtained value was added to the average value to obtain the correction factor. The OD-value of each peptide was then divided by this correction factor. If a value of 0.9 or higher was found, then relevant binding was considered to be present between the specific peptide and the respective serum. Particularly, domains (response of clustering of reactive peptides reactive with several individual sera) comprising several relevant peptides were claimed in the present invention. These domains are indicated (colored grey) in the above-mentioned tables.

Any of the above peptides could form the basis for diagnostic kits comprising the peptides, vaccines (as peptide, DNA, or vector vaccine) or for raising neutralizing antibodies to treat and/or prevent SARS.

TABLE 1
Binding of the sera called SARS-yellow, SARS-green,
1a, 1b, 2, 6, 37, 62 and London to linear peptides of Orf9 of SARS-CoV TOR2.

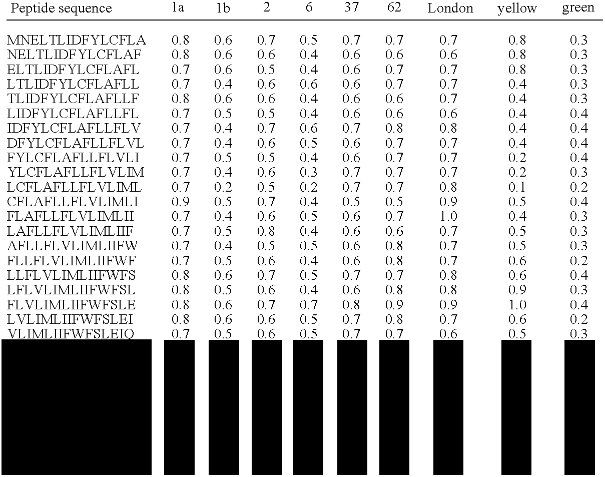

TABLE 2
Binding of the sera called SARS-yellow, SARS-green,
1a, 1b, 2, 6, 37, 62 and London to looped/cyclic peptides of Orf9 of SARS-CoV TOR2.

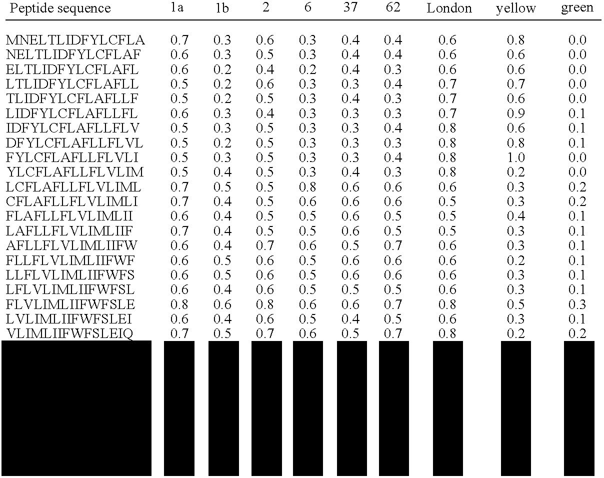

TABLE 3
Binding of the sera called SARS-yellow, SARS-green,
1a, 1b, 2, 6, 37, 62 and London to linear peptides of Orf10 of SARS-CoV TOR2.

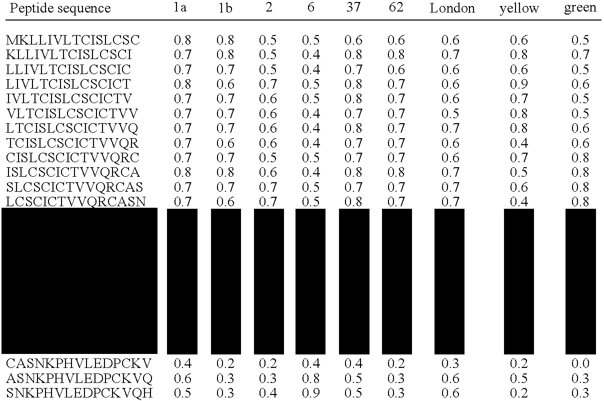

TABLE 4
Binding of the sera called SARS-yellow, SARS-green,
1a, 1b, 2, 6, 37, 62 and London to looped/cyclic peptides of Orf10 of SARS-CoV TOR2.

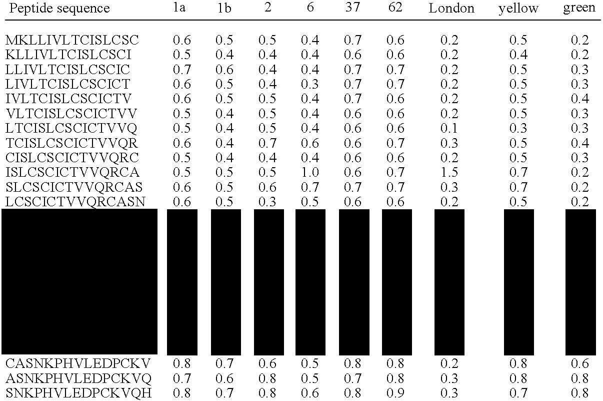

TABLE 5
Binding of the sera called SARS-yellow, SARS-green,
1a, 1b, 2, 6, 37, 62 and London to linear peptides of Orf13 of SARS-CoV TOR2.

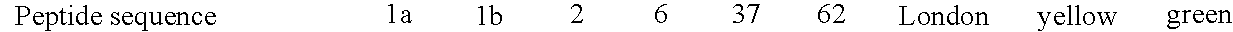

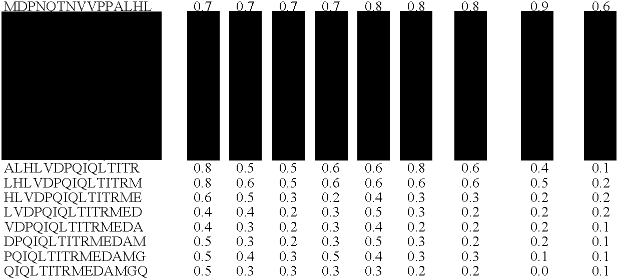

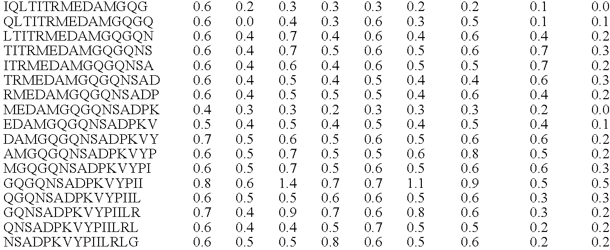

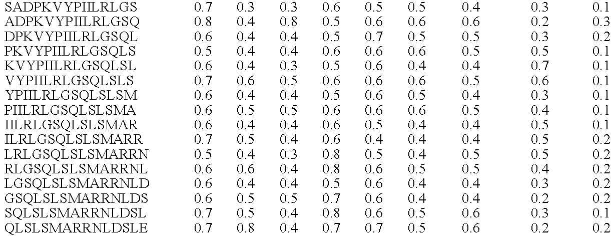

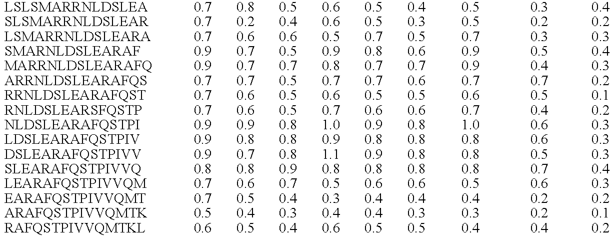

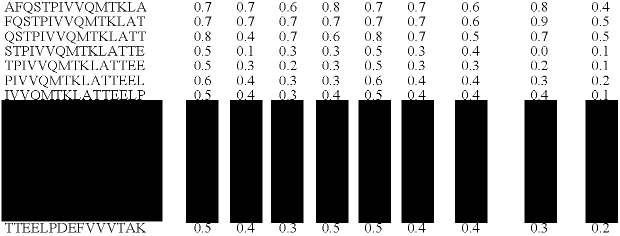

TABLE 6
Binding of the sera called SARS-yellow, SARS-green,
1a, 1b, 2, 6, 37, 62 and London to looped/cyclic peptides of Orf13 of SARS-CoV TOR2.

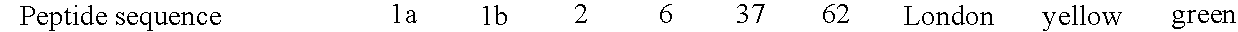

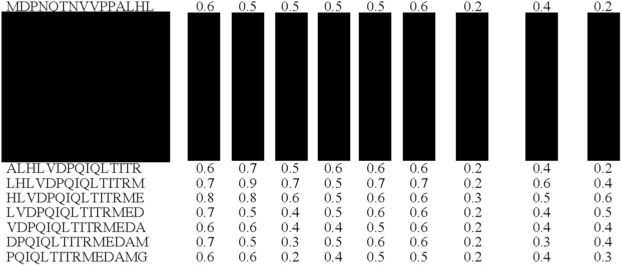

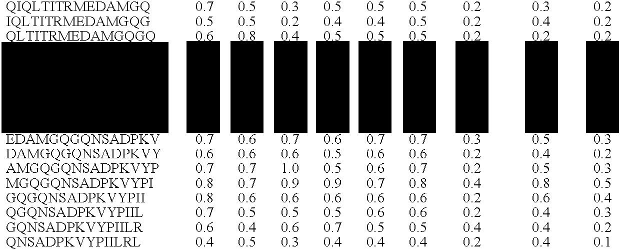

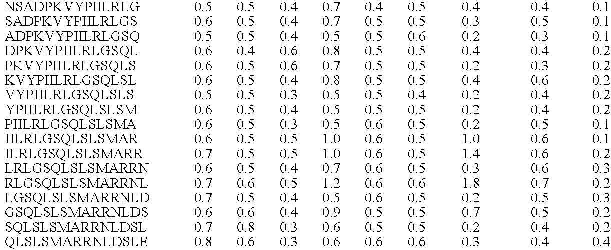

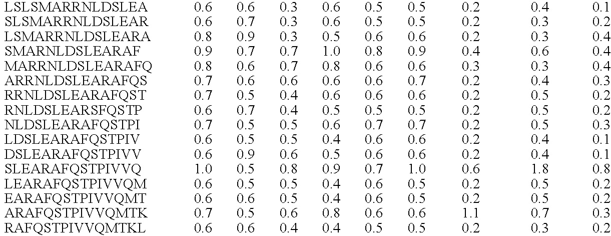

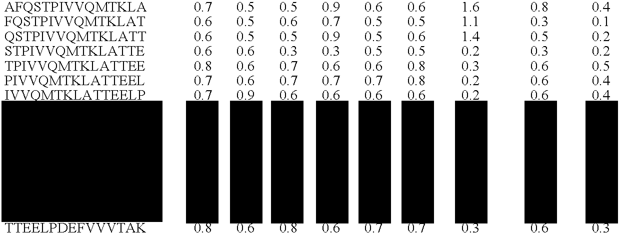

TABLE 7
Binding of the sera called SARS-yellow, SARS-green, 1a, 1b,
2, 6, 37, 62 and London to linear peptides of Orf14 of SARS-CoV TOR2.

| Peptide sequence | 1a | 1b | 2 | 6 | 37 | 62 | London | yellow | green |
|---|---|---|---|---|---|---|---|---|---|
| MLPPCYNFLKEQHCQ | 0.4 | 0.4 | 0.3 | 0.5 | 0.5 | 0.3 | 0.4 | 0.2 | 0.2 |
| LPPCYNFLKEQHCQK | 0.3 | 0.2 | 0.2 | 0.2 | 0.3 | 0.2 | 0.1 | 0.0 | 0.0 |
| PPCYNFLKEQHCQKA | 0.3 | 0.2 | 0.1 | 0.2 | 0.3 | 0.2 | 0.1 | 0.0 | 0.1 |
| PCYNFLKEQHCQKAS | 0.3 | 0.2 | 0.1 | 0.3 | 0.4 | 0.1 | 0.2 | 0.1 | 0.1 |
| CYNFLKEQHCQKAST | 0.5 | 0.3 | 0.5 | 0.5 | 0.6 | 0.4 | 0.7 | 0.1 | 0.4 |
| YNFLKEQHCQKASTQ | 0.5 | 0.4 | 0.5 | 0.5 | 0.6 | 0.5 | 0.7 | 0.3 | 0.5 |
| NFLKEQHCQKASTQR | 0.4 | 0.3 | 0.4 | 0.4 | 0.5 | 0.3 | 0.4 | 0.3 | 0.3 |
| FLKEQHCQKASTQRE | 0.3 | 0.3 | 0.3 | 0.2 | 0.4 | 0.3 | 0.3 | 0.2 | 0.1 |
| LKEQHCQKASTQREA | 0.3 | 0.2 | 0.4 | 0.2 | 0.4 | 0.2 | 0.3 | 0.3 | 0.1 |
| KEQHCQKASTQREAE | 0.4 | 0.3 | 0.4 | 0.3 | 0.4 | 0.2 | 0.3 | 0.2 | 0.1 |
| EQHCQKASTQREAEA | 0.4 | 0.4 | 0.6 | 0.4 | 0.5 | 0.3 | 0.5 | 0.4 | 0.3 |
| QHCQKASTQREAEAA | 0.4 | 0.4 | 0.6 | 0.3 | 0.5 | 0.4 | 0.5 | 0.3 | 0.3 |
| HCQKASTQREAEAAV | 0.6 | 0.5 | 0.9 | 0.6 | 0.7 | 0.7 | 0.7 | 0.6 | 0.4 |
| CQKASTQREAEAAVK | 0.3 | 0.2 | 0.3 | 0.2 | 0.3 | 0.2 | 0.2 | 0.1 | 0.0 |
| QKASTQREAEAAVKP | 0.4 | 0.3 | 0.4 | 0.3 | 0.4 | 0.3 | 0.3 | 0.1 | 0.1 |
| KASTQREAEAAVKPL | 0.3 | 0.3 | 0.3 | 0.3 | 0.3 | 0.2 | 0.2 | 0.1 | 0.1 |
| ASTQREAEAAVKPLL | 0.4 | 0.3 | 0.4 | 0.5 | 0.5 | 0.4 | 0.4 | 0.4 | 0.4 |
| STQREAEAAVKPLLA | 0.5 | 0.3 | 0.5 | 0.5 | 0.6 | 0.4 | 0.4 | 0.5 | 0.4 |
| TQREAEAAVKPLLAP | 0.4 | 0.2 | 0.4 | 0.4 | 0.4 | 0.3 | 0.4 | 0.2 | 0.3 |
| QREAEAAVKPLLAPH | 0.4 | 0.3 | 0.4 | 0.5 | 0.4 | 0.3 | 0.4 | 0.2 | 0.3 |
| REAEAAVKPLLAPHH | 0.5 | 0.3 | 0.4 | 0.3 | 0.5 | 0.3 | 0.4 | 0.2 | 0.1 |
| EAEAAVKPLLAPHHV | 0.5 | 0.3 | 0.7 | 0.4 | 0.5 | 0.4 | 0.6 | 0.2 | 0.4 |
| AEAAVKPLLAPHHVV | 0.7 | 0.5 | 1.3 | 0.6 | 0.8 | 0.8 | 0.8 | 0.8 | 0.8 |
| EAAVKPLLAPHHVVA | 0.6 | 0.5 | 0.7 | 0.5 | 0.7 | 0.6 | 0.8 | 0.6 | 0.6 |
| AAVKPLLAPHHVVAV | 0.8 | 0.6 | 1.0 | 0.7 | 0.9 | 0.9 | 0.8 | 0.8 | 0.6 |
| AVKPLLAPHHVVAVI | 0.6 | 0.4 | 0.5 | 0.5 | 0.7 | 0.5 | 0.5 | 0.6 | 0.3 |
| VKPLLAPHHVVAVIQ | 0.6 | 0.5 | 0.6 | 0.5 | 0.7 | 0.5 | 0.6 | 0.4 | 0.3 |
| KPLLAPHHVVAVIQE | 0.5 | 0.4 | 0.4 | 0.4 | 0.6 | 0.4 | 0.4 | 0.4 | 0.3 |
| PLLAPHHVVAVIQEI | 0.5 | 0.4 | 0.4 | 0.4 | 0.6 | 0.4 | 0.5 | 0.4 | 0.2 |
| LLAPHHVVAVIQEIQ | 0.5 | 0.5 | 0.5 | 0.5 | 0.6 | 0.5 | 0.5 | 0.5 | 0.3 |
| LAPHHVVAVIQEIQL | 0.5 | 0.4 | 0.4 | 0.4 | 0.5 | 0.4 | 0.4 | 0.4 | 0.1 |
| APHHVVAVIQEIQLL | 0.5 | 0.4 | 0.4 | 0.4 | 0.5 | 0.3 | 0.3 | 0.3 | 0.1 |
| PHHVVAVIQEIQLLA | 0.4 | 0.3 | 0.4 | 0.4 | 0.5 | 0.3 | 0.3 | 0.4 | 0.2 |
| HHVVAVIQEIQLLAA | 0.4 | 0.4 | 0.4 | 0.4 | 0.5 | 0.3 | 0.4 | 0.4 | 0.2 |
| HVVAVIQEIQLLAAV | 0.5 | 0.4 | 0.5 | 0.4 | 0.6 | 0.3 | 0.4 | 0.4 | 0.1 |
| VVAVIQEIQLLAAVG | 0.8 | 0.7 | 0.7 | 1.3 | 0.6 | 0.8 | 1.0 | 0.4 | 0.5 |
| VAVIQEIQLLAAVGE | 0.5 | 0.4 | 0.3 | 0.4 | 0.5 | 0.3 | 0.3 | 0.2 | 0.3 |
| AVIQEIQLLAAVGEI | 2.1 | 2.4 | 1.7 | 2.0 | 1.7 | 2.1 | 1.7 | 2.3 | 2.5 |
| VIQEIQLLAAVGEIL | 0.5 | 0.4 | 0.4 | 0.3 | 0.6 | 0.4 | 0.4 | 0.3 | 0.5 |
| IQEIQLLAAVGEILL | 0.5 | 0.4 | 0.4 | 0.4 | 0.7 | 0.4 | 0.3 | 0.4 | 0.3 |
| QEIQLLAAVGEILLL | 0.5 | 0.4 | 0.5 | 0.4 | 0.6 | 0.4 | 0.3 | 0.5 | 0.2 |
| EIQLLAAVGEILLLE | 0.6 | 0.5 | 0.5 | 0.4 | 0.6 | 0.4 | 0.4 | 0.5 | 0.3 |
| IQLLAAVGEILLLEW | 0.5 | 0.4 | 0.5 | 0.4 | 0.6 | 0.4 | 0.4 | 0.4 | 0.2 |
| QLLAAVGEILLLEWL | 0.5 | 0.4 | 0.4 | 0.3 | 0.6 | 0.4 | 0.4 | 0.4 | 0.1 |
| LLAAVGEILLLEWLA | 0.5 | 0.4 | 0.4 | 0.4 | 0.6 | 0.4 | 0.4 | 0.4 | 0.1 |
| LAAVGEILLLEWLAE | 0.6 | 0.5 | 0.5 | 0.5 | 0.7 | 0.5 | 0.6 | 0.6 | 0.5 |
| AAVGEILLLEWLAEV | 0.6 | 0.5 | 0.6 | 0.5 | 0.8 | 0.6 | 0.6 | 0.6 | 0.5 |
| AVGEILLLEWLAEVV | 0.6 | 0.5 | 0.5 | 0.5 | 0.7 | 0.6 | 0.6 | 0.5 | 0.3 |
| VGEILLLEWLAEVVK | 0.5 | 0.4 | 0.4 | 0.5 | 0.6 | 0.4 | 0.5 | 0.4 | 0.2 |
| GEILLLEWLAEVVKL | 0.5 | 0.4 | 0.4 | 0.5 | 0.6 | 0.5 | 0.6 | 0.3 | 0.3 |
| EILLLEWLAEVVKLP | 0.5 | 0.5 | 0.5 | 0.7 | 0.6 | 0.5 | 0.7 | 0.4 | 0.4 |
| ILLLEWLAEVVKLPS | 0.5 | 0.4 | 0.5 | 0.5 | 0.6 | 0.4 | 0.5 | 0.3 | 0.1 |
| LLLEWLAEVVKLPSR | 0.5 | 0.4 | 0.4 | 0.6 | 0.7 | 0.4 | 0.6 | 0.2 | 0.3 |
| LLEWLAEVVKLPSRY | 0.5 | 0.4 | 0.3 | 0.5 | 0.5 | 0.4 | 0.4 | 0.3 | 0.1 |
| LEWLAEVVKLPSRYC | 0.6 | 0.3 | 0.4 | 0.4 | 0.7 | 0.4 | 0.5 | 0.2 | 0.1 |
| EWLAEVVKLPSRYCC | 0.8 | 0.6 | 0.7 | 0.6 | 1.0 | 0.7 | 1.0 | 0.3 | 0.6 |

TABLE 8
Binding of the sera called SARS-yellow, SARS-green,
1a, 1b, 2, 6, 37, 62 and London to looped/cyclic peptides of Orf14 of SARS-CoV TOR2.

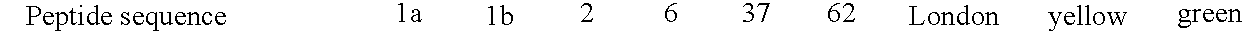

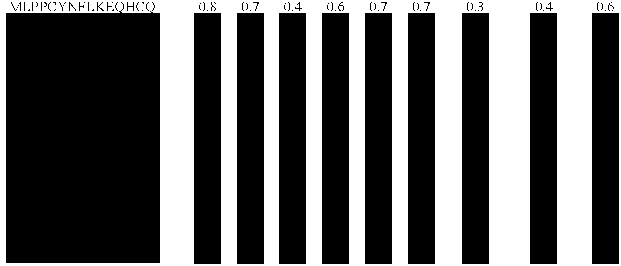

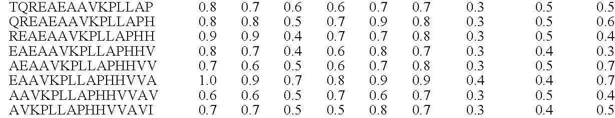

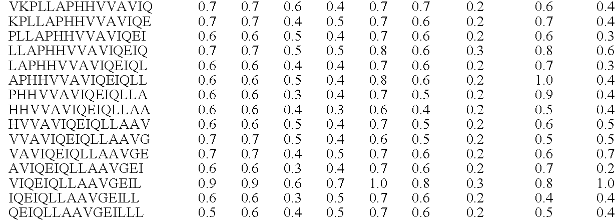

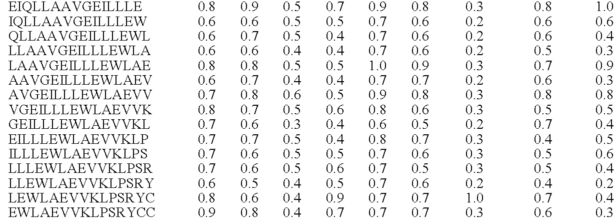

TABLE 9
Binding of two control sera to linear and looped/cyclic peptides of Orf9 of SARS-CoV
TOR2.

| | Control serum | Control serum | Control serum | Control Serum |
|---|---|---|---|---|
| | LUMC linear | Blood-bank | LUMC looped | Blood-Bank |
| Peptide sequence | peptides | linear peptides | peptides | Looped peptides |
| MNELTLIDFYLCFLA | 1.1 | 0.7 | 0.9 | 0.5 |
| NELTLIDFYLCFLAF | 0.6 | 0.6 | 0.9 | 0.5 |
| ELTLIDFYLCFLAFL | 0.5 | 0.7 | 0.7 | 0.6 |
| LTLIDFYLCFLAFLL | 0.8 | 0.9 | 0.7 | 0.5 |
| TLIDFYLCFLAFLLF | 0.8 | 0.7 | 0.8 | 0.4 |
| LIDFYLCFLAFLLFL | 0.4 | 0.7 | 0.7 | 0.5 |
| IDFYLCFLAFLLFLV | 0.5 | 0.9 | 0.7 | 0.6 |
| DFYLCFLAFLLFLVL | 0.4 | 0.7 | 0.6 | 0.6 |
| FYLCFLAFLLFLVLI | 0.4 | 0.8 | 0.6 | 0.7 |
| YLCFLAFLLFLVLIM | 0.4 | 0.8 | 0.8 | 0.6 |
| LCFLAFLLFLVLIML | 0.4 | 0.7 | 0.4 | 0.7 |
| CFLAFLLFLVLIMLI | 0.3 | 0.6 | 0.4 | 0.8 |
| FLAFLLFLVLIMLII | 0.5 | 0.7 | 0.4 | 0.7 |
| LAFLLFLVLIMLIIF | 0.3 | 0.6 | 0.4 | 0.8 |
| AFLLFLVLIMLIIFW | 0.4 | 0.8 | 0.4 | 0.8 |
| FLLFLVLIMLIIFWF | 0.3 | 0.9 | 0.5 | 0.6 |
| LLFLVLIMLIIFWFS | 0.4 | 0.9 | 0.4 | 0.9 |
| LFLVLIMLIIFWFSL | 0.4 | 0.8 | 0.4 | 0.8 |
| FLVLIMLIIFWFSLE | 0.7 | 0.9 | 0.7 | 0.8 |
| LVLIMLIIFWFSLEI | 0.8 | 0.9 | 0.3 | 0.7 |
| VLIMLIIFWFSLEIQ | 0.7 | 0.8 | 0.4 | 0.7 |
| LIMLIIFWFSLEIQD | 1.1 | 0.9 | 0.7 | 0.7 |
| IMLIIFWFSLEIQDL | 0.7 | 0.8 | 0.4 | 0.6 |
| MLIIFWFSLEIQDLE | 0.9 | 1.0 | 0.8 | 0.7 |
| LIIFWFSLEIQDLEE | 0.7 | 0.8 | 0.9 | 0.7 |
| IIFWFSLEIQDLEEP | 0.7 | 0.9 | 0.8 | 0.9 |
| IFWFSLEIQDLEEPC | 0.7 | 0.8 | 0.8 | 0.8 |
| FWFSLEIQDLEEPCT | 0.3 | 0.5 | 0.8 | 1.1 |
| WFSLEIQDLEEPCTK | 0.5 | 0.6 | 0.5 | 0.7 |
| FSLEIQDLEEPCTKV | 0.6 | 0.7 | 0.7 | 1.0 |

TABLE 10
Binding of two control sera to linear and looped/cyclic peptides of Orf10 of SARS-CoV TOR2.

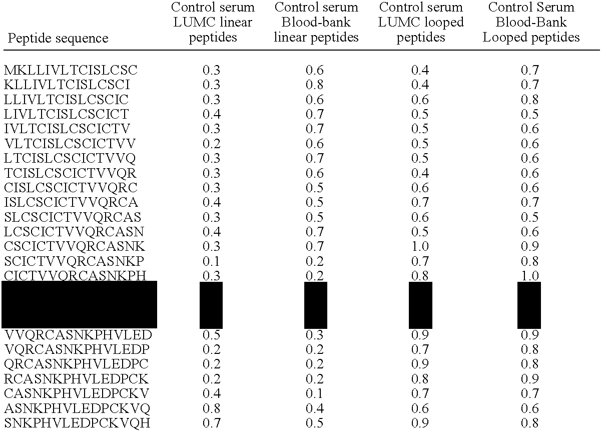

TABLE 11
Binding of two control sera to linear and looped/cyclic peptides of Orf13 of SARS-CoV TOR2.

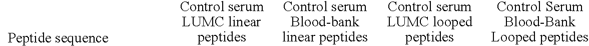

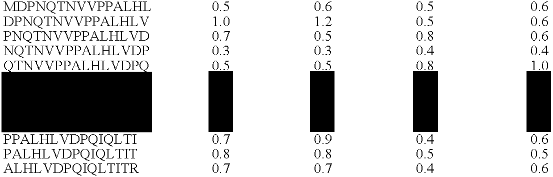

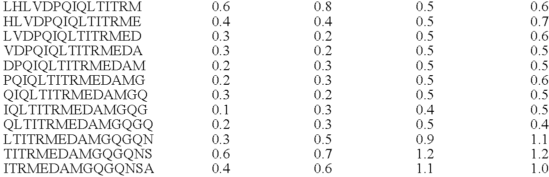

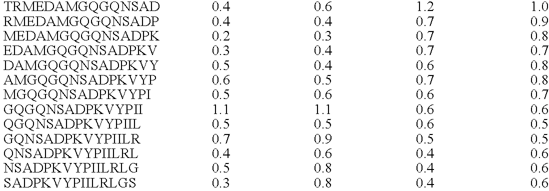

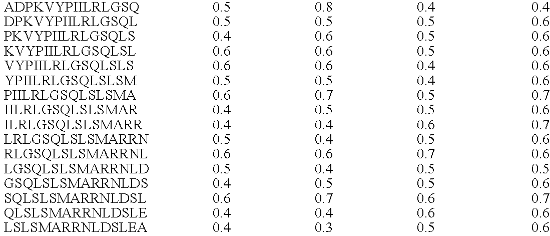

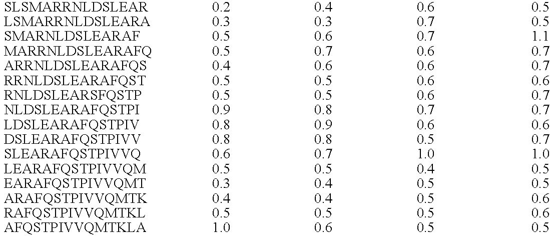

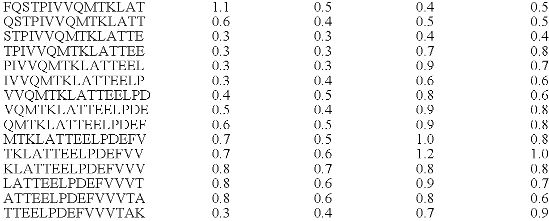

TABLE 12
Binding of two control sera to linear and looped/cyclic peptides of Orf14 of
SARS-CoV TOR2.

| | Control serum | Control serum | Control serum | Control Serum |
|---|---|---|---|---|
| | LUMC linear | Blood-bank | LUMC looped | Blood-Bank |
| Peptide sequence | peptides | linear peptides | peptides | Looped peptides |
| MLPPCYNFLKEQHCQ | 0.5 | 0.4 | 0.6 | 0.7 |
| LPPCYNFLKEQHCQK | 0.2 | 0.2 | 0.6 | 0.9 |
| PPCYNFLKEQHCQKA | 0.1 | 0.2 | 0.6 | 0.8 |
| PCYNFLKEQHCQKAS | 0.1 | 0.0 | 0.5 | 0.7 |
| CYNFLKEQHCQKAST | 0.3 | 0.4 | 0.5 | 0.6 |
| YNFLKEQHCQKASTQ | 0.5 | 0.5 | 0.5 | 0.7 |
| NFLKEQHCQKASTQR | 0.7 | 0.5 | 0.7 | 0.6 |
| FLKEQHCQKASTQRE | 0.3 | 0.4 | 1.1 | 0.9 |
| LKEQHCQKASTQREA | 0.3 | 0.3 | 0.9 | 0.9 |
| KEQHCQKASTQREAE | 0.3 | 0.3 | 0.8 | 0.9 |
| EQHCQKASTQREAEA | 0.4 | 0.5 | 0.8 | 0.9 |
| QHCQKASTQREAEAA | 0.4 | 0.4 | 1.2 | 1.1 |
| HCQKASTQREAEAAV | 0.6 | 0.5 | 0.8 | 0.9 |
| CQKASTQREAEAAVK | 0.2 | 0.2 | 0.5 | 0.7 |
| QKASTQREAEAAVKP | 0.3 | 0.3 | 0.8 | 0.7 |
| KASTQREAEAAVKPL | 0.3 | 0.3 | 0.6 | 0.6 |
| ASTQREAEAAVKPLL | 0.4 | 0.5 | 0.8 | 0.9 |
| STQREAEAAVKPLLA | 0.6 | 0.6 | 1.2 | 1.2 |
| TQREAEAAVKPLLAP | 0.4 | 0.4 | 0.7 | 0.8 |
| QREAEAAVKPLLAPH | 0.4 | 0.4 | 0.7 | 0.7 |
| REAEAAVKPLLAPHH | 0.3 | 0.2 | 0.6 | 0.7 |
| EAEAAVKPLLAPHHV | 0.4 | 0.4 | 0.6 | 0.7 |
| AEAAVKPLLAPHHVV | 1.0 | 0.8 | 0.6 | 0.6 |
| EAAVKPLLAPHHVVA | 0.8 | 0.6 | 0.7 | 0.8 |
| AAVKPLLAPHHVVAV | 1.0 | 1.0 | 0.7 | 0.6 |
| AVKPLLAPHHVVAVI | 0.6 | 0.7 | 0.7 | 0.7 |
| VKPLLAPHHVVAVIQ | 0.6 | 0.7 | 0.6 | 0.7 |
| KPLLAPHHVVAVIQE | 0.6 | 0.5 | 0.6 | 0.6 |
| PLLAPHHVVAVIQEI | 0.5 | 0.4 | 0.6 | 0.6 |
| LLAPHHVVAVIQEIQ | 0.6 | 0.4 | 0.8 | 0.8 |
| LAPHHVVAVIQEIQL | 0.6 | 0.3 | 0.5 | 0.5 |
| APHHVVAVIQEIQLL | 0.6 | 0.3 | 0.5 | 0.6 |
| PHHVVAVIQEIQLLA | 0.5 | 0.4 | 0.6 | 0.5 |
| HHVVAVIQEIQLLAA | 0.4 | 0.4 | 0.4 | 0.5 |
| HVVAVIQEIQLLAAV | 0.5 | 0.4 | 0.4 | 0.6 |
| VVAVIQEIQLLAAVG | 1.1 | 1.1 | 0.4 | 0.5 |
| VAVIQEIQLLAAVGE | 0.3 | 0.3 | 0.6 | 0.6 |
| AVIQEIQLLAAVGEI | 1.4 | 1.8 | 0.5 | 0.5 |
| VIQEIQLLAAVGEIL | 0.6 | 0.4 | 0.9 | 0.5 |
| IQEIQLLAAVGEILL | 0.6 | 0.4 | 0.5 | 0.6 |
| QEIQLLAAVGEILLL | 0.7 | 0.4 | 0.5 | 0.6 |
| EIQLLAAVGEILLLE | 0.8 | 0.4 | 0.7 | 0.7 |
| IQLLAAVGEILLLEW | 0.6 | 0.5 | 0.5 | 0.6 |
| QLLAAVGEILLLEWL | 0.6 | 0.4 | 0.5 | 0.6 |
| LLAAVGEILLLEWLA | 0.6 | 0.5 | 0.6 | 0.6 |
| LAAVGEILLLEWLAE | 0.6 | 0.4 | 0.7 | 0.7 |
| AAVGEILLLEWLAEV | 0.6 | 0.4 | 0.7 | 0.7 |
| AVGEILLLEWLAEVV | 0.8 | 0.5 | 0.8 | 0.8 |
| VGEILLLEWLAEVVK | 0.6 | 0.5 | 0.8 | 0.7 |
| GEILLLEWLAEVVKL | 0.5 | 0.6 | 0.5 | 0.5 |
| EILLLEWLAEVVKLP | 0.6 | 0.7 | 0.6 | 0.7 |
| ILLLEWLAEVVKLPS | 0.5 | 0.6 | 0.6 | 0.7 |
| LLLEWLAEVVKLPSR | 0.5 | 0.7 | 0.5 | 0.7 |
| LLEWLAEVVKLPSRY | 0.4 | 0.5 | 0.6 | 0.6 |
| LEWLAEVVKLPSRYC | 0.4 | 0.4 | 0.7 | 0.8 |
| EWLAEVVKLPSRYCC | 0.6 | 0.7 | 0.6 | 0.7 |

TABLE 13
Binding of a rabbit serum to linear and
looped/cyclic peptides of Orf9 of SARS-CoV TOR2.

| | Rabbit serum | Rabbit serum |
|---|---|---|
| Peptide sequence | linear peptides | looped peptides |
| MNELTLIDFYLCFLA | 0.4 | 0.5 |
| NELTLIDFYLCFLAF | 0.4 | 0.8 |
| ELTLIDFYLCFLAFL | 0.4 | 0.6 |
| LTLIDFYLCFLAFLL | 0.3 | 0.5 |
| TLIDFYLCFLAFLLF | 0.4 | 0.4 |
| LIDFYLCFLAFLLFL | 0.3 | 0.3 |
| IDFYLCFLAFLLFLV | 0.4 | 0.5 |
| DFYLCFLAFLLFLVL | 0.3 | 0.4 |
| FYLCFLAFLLFLVLI | 0.3 | 0.6 |
| YLCFLAFLLFLVLIM | 0.3 | 0.3 |
| LCFLAFLLFLVLIML | 0.2 | 0.3 |
| CFLAFLLFLVLIMLI | 0.0 | 0.6 |
| FLAFLLFLVLIMLII | 0.3 | 0.7 |
| LAFLLFLVLIMLIIF | 0.4 | 1.1 |
| AFLLFLVLIMLIIFW | 0.5 | 1.0 |
| FLLFLVLIMLIIFWF | 0.5 | 0.7 |
| LLFLVLIMLIIFWFS | 0.5 | 0.6 |
| LFLVLIMLIIFWFSL | 0.4 | 0.5 |
| FLVLIMLIIFWFSLE | 0.4 | 0.6 |
| LVLIMLIIFWFSLEI | 0.4 | 0.6 |
| VLIMLIIFWFSLEIQ | 0.4 | 0.2 |
| LIMLIIFWFSLEIQD | 0.4 | 0.6 |
| IMLIIFWFSLEIQDL | 0.9 | 0.1 |
| MLIIFWFSLEIQDLE | 1.7 | 0.5 |
| LIIFWFSLEIQDLEE | 0.5 | 0.7 |
| IIFWFSLEIQDLEEP | 0.4 | 0.0 |
| IFWFSLEIQDLEEPC | 0.3 | 0.5 |
| FWFSLEIQDLEEPCT | 0.1 | 0.5 |
| WFSLEIQDLEEPCTK | 0.0 | 0.4 |
| FSLEIQDLEEPCTKV | 0.4 | 0.5 |

TABLE 14
Binding of a rabbit serum to linear and looped/cyclic
peptides of Orf10 of SARS-CoV TOR2.

| | Rabbit | Rabbit |
|---|---|---|
| Peptide sequence | serum linear peptides | serum looped peptides |
| MKLLIVLTCISLCSC | 0.1 | 0.5 |
| KLLIVLTCISLCSCI | 0.0 | 0.5 |
| LLIVLTCISLCSCIC | 0.1 | 0.5 |
| LIVLTCISLCSCICT | 0.1 | 0.5 |
| IVLTCISLCSCICTV | 0.1 | 0.6 |
| VLTCISLCSCICTVV | 0.0 | 0.4 |
| LTCISLCSCICTVVQ | 0.1 | 0.2 |
| TCISLCSCICTVVQR | 0.0 | 0.5 |
| CISLCSCICTVVQRC | 0.0 | 0.5 |
| ISLCSCICTVVQRCA | 0.1 | 0.5 |
| SLCSCICTVVQRCAS | 0.1 | 0.2 |
| LCSCICTVVQRCASN | 0.1 | 1.1 |
| CSCICTVVQRCASNK | 0.1 | 0.0 |
| SCICTVVQRCASNKP | 0.0 | 0.8 |
| CICTVVQRCASNKPH | 0.0 | 0.5 |
| ICTVVQRCASNKPHV | 0.3 | 0.4 |
| CTVVQRCASNKPHVL | 1.7 | 1.0 |
| TVVQRCASNKPHVLE | 0.7 | 0.3 |
| VVQRCASNKPHVLED | 0.2 | 0.5 |
| VQRCASNKPHVLEDP | 0.1 | 0.5 |
| QRCASNKPHVLEDPC | 0.1 | 0.5 |
| RCASNKPHVLEDPCK | 0.1 | 0.6 |
| CASNKPHVLEDPCKV | 0.5 | 0.8 |
| ASNKPHVLEDPCKVQ | 0.8 | 0.9 |
| SNKPHVLEDPCKVQH | 0.8 | 0.4 |

TABLE 15
Binding of a rabbit serum to linear and looped/cyclic
peptides of Orf13 of SARS-CoV TOR2.

| | Rabbit serum | Rabbit |
|---|---|---|
| Peptide sequence | linear peptides | serum looped peptides |
| MDPNQTNVVPPALHL | 1.1 | 0.1 |
| DPNQTNVVPPALHLV | 2.3 | 0.2 |
| PNQTNVVPPALHLVD | 0.4 | 0.2 |
| NQTNVVPPALHLVDP | 0.1 | 0.4 |
| QTNVVPPALHLVDPQ | 0.1 | 0.4 |
| TNVVPPALHLVDPQI | 0.1 | 0.5 |
| NVVPPALHLVDPQIQ | 0.1 | 0.5 |
| VVPPALHLVDPQIQL | 1.2 | 0.4 |
| VPPALHLVDPQIQLT | 1.0 | 0.4 |
| PPALHLVDPQIQLTI | 0.2 | 0.3 |
| PALHLVDPQIQLTIT | 1.3 | 0.4 |
| ALHLVDPQIQLTITR | 0.5 | 0.5 |
| LHLVDPQIQLTITRM | 0.5 | 0.6 |
| HLVDPQIQLTITRME | 0.1 | 0.3 |
| LVDPQIQLTITRMED | 0.1 | 0.2 |
| VDPQIQLTITRMEDA | 0.0 | 0.4 |
| DPQIQLTITRMEDAM | 0.1 | 0.3 |
| PQIQLTITRMEDAMG | 0.0 | 0.2 |
| QIQLTITRMEDAMGQ | 0.1 | 0.2 |
| IQLTITRMEDAMGQG | 0.0 | 0.2 |
| QLTITRMEDAMGQGQ | 0.1 | 0.4 |
| LTITRMEDAMGQGQN | 0.3 | 0.3 |
| TITRMEDAMGQGQNS | 0.1 | 0.6 |
| ITRMEDAMGQGQNSA | 0.1 | 0.4 |
| TRMEDAMGQGQNSAD | 0.4 | 0.4 |
| RMEDAMGQGQNSADP | 0.1 | 0.5 |
| MEDAMGQGQNSADPK | 0.1 | 0.4 |
| EDAMGQGQNSADPKV | 0.6 | 0.5 |
| DAMGQGQNSADPKVY | 0.3 | 0.4 |
| AMGQGQNSADPKVYP | 0.1 | 0.6 |
| MGQGQNSADPKVYPI | 0.1 | 0.6 |
| GQGQNSADPKVYPII | 0.4 | 0.5 |
| QGQNSADPKVYPIIL | 0.1 | 0.5 |
| GQNSADPKVYPIILR | 0.1 | 0.6 |
| QNSADPKVYPIILRL | 0.1 | 0.1 |
| NSADPKVYPIILRLG | 0.1 | 0.4 |
| SADPKVYPIILRLGS | 0.1 | 0.4 |
| ADPKVYPIILRLGSQ | 0.4 | 0.3 |
| DPKVYPIILRLGSQL | 0.1 | 0.4 |
| PKVYPIILRLGSQLS | 0.1 | 0.4 |
| KVYPIILRLGSQLSL | 1.8 | 0.5 |
| VYPIILRLGSQLSLS | 0.6 | 1.0 |
| YPIILRLGSQLSLSM | 0.2 | 0.4 |
| PIILRLGSQLSLSMA | 0.1 | 0.5 |
| IILRLGSQLSLSMAR | 0.6 | 0.5 |
| ILRLGSQLSLSMARR | 0.1 | 0.7 |
| LRLGSQLSLSMARRN | 0.1 | 0.5 |
| RLGSQLSLSMARRNL | 0.1 | 2.5 |
| LGSQLSLSMARRNLD | 0.1 | 0.3 |
| GSQLSLSMARRNLDS | 0.1 | 0.7 |
| SQLSLSMARRNLDSL | 0.1 | 0.4 |
| QLSLSMARRNLDSLE | 0.1 | 0.3 |
| LSLSMARRNLDSLEA | 0.1 | 0.3 |
| SLSMARRNLDSLEAR | 0.1 | 0.4 |
| LSMARRNLDSLEARA | 0.0 | 0.4 |
| SMARRNLDSLEARAF | 0.1 | 0.4 |
| MARRNLDSLEARAFQ | 0.1 | 0.5 |
| ARRNLDSLEARAFQS | 0.1 | 0.5 |
| RRNLDSLEARAFQST | 0.3 | 0.5 |
| RNLDSLEARAFQSTP | 0.3 | 0.5 |
| NLDSLEARAFQSTPI | 0.2 | 0.4 |
| LDSLEARAFQSTPIV | 0.1 | 0.5 |
| DSLEARAFQSTPIVV | 0.1 | 0.5 |
| SLEARAFQSTPIVVQ | 0.1 | 0.6 |
| LEARAFQSTPIVVQM | 0.1 | 0.5 |
| EARAFQSTPIVVQMT | 0.1 | 0.4 |
| ARAFQSTPIVVQMTK | 0.2 | 0.7 |
| RAFQSTPIVVQMTKL | 0.1 | 0.7 |
| AFQSTPIVVQMTKLA | 0.1 | 0.5 |
| FQSTPIVVQMTKLAT | 0.1 | 0.6 |
| QSTPIVVQMTKLATT | 0.0 | 0.6 |
| STPIVVQMTKLATTE | 0.1 | 0.5 |
| TPIVVQMTKLATTEE | 0.1 | 0.4 |
| PIVVQMTKLATTEEL | 0.1 | 0.6 |
| IVVQMTKLATTEELP | 0.2 | 0.4 |
| VVQMTKLATTEELPD | 0.3 | 0.6 |
| VQMTKLATTEELPDE | 0.3 | 0.6 |
| QMTKLATTEELPDEF | 0.1 | 0.6 |
| MTKLATTEELPDEFV | 0.1 | 0.6 |
| TKLATTEELPDEFVV | 0.1 | 0.6 |
| KLATTEELPDEFVVV | 0.2 | 0.5 |
| LATTEELPDEFVVVT | 0.1 | 0.6 |
| ATTEELPDEFVVVTA | 0.1 | 0.5 |
| TTEELPDEFVVVTAK | 0.0 | 0.5 |

TABLE 16
Binding of a rabbit serum to linear and
looped/cyclic peptides of Orf14 of SARS-CoV TOR2.

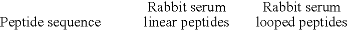

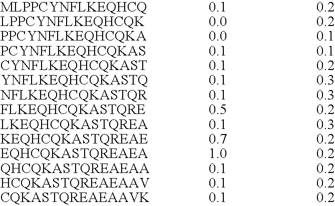

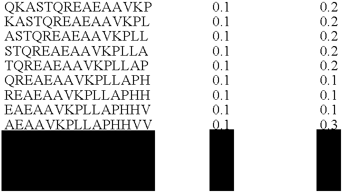

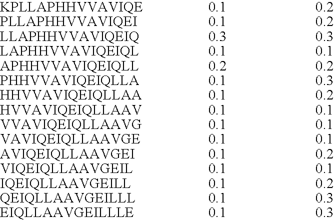

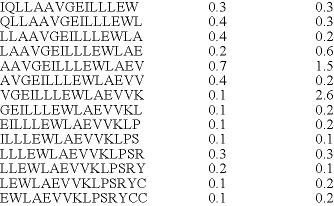

### REFERENCES

Holmes K. V. (2003) SARS coronavirus: a new challenge for prevention and therapy. J. Clin. Invest. 111, 1605-1609.
Ksiazek T. G., et al. (2003) A novel coronavirus associated with severe acute respiratory syndrome. N. Eng. J. Med. 348, 1953-1966.
Marra M. A., et al. (2003) The genome sequence of the SARS-associated coronavirus. Science 300, 1399-1404.
Rota P. A., et al. (2003) Characterization of a novel coronavirus associated with severe acute respiratory syndrome. Science 300, 1394-1399.
Slootstra J. W., et al. (1996) Structural aspects of antibody-antigen interaction revealed through small random peptide libraries. Mol. Divers. 1, 87-96.

1. An isolated peptide having an amino acid sequence selected from the group consisting of SEQ ID NO:11-SEQ ID NO:80 and SEQ ID NO:81.

2. A second peptide, wherein said second peptide consists of at least two peptides of claim 1.

3. A part of the peptide of claim 1, wherein the part is recognized by antibodies present in serum derived from a subject that has been immunized against Severe Acute Respiratory Syndrome Coronavirus (SARS-Co-V).

4. An analogue of the peptide of claim 1, wherein said analogue has an amino acid sequence consisting of the amino acid sequence of the peptide with one or more amino acid substitutions in the peptide, and said analogue is recognized by antibodies present in serum derived from a subject that has been immunized with Severe Acute Respiratory Syndrome Coronavirus (SARS-Co-V).

5. A fusion protein or a conjugate comprising the isolated peptide of claim 1.

6. An isolated nucleic acid molecule encoding the peptide of claim 1.

7. An isolated antibody, wherein said antibody is able to specifically recognize the peptide claim 1.

8. The isolated antibody of claim 7, wherein said antibody is a monoclonal antibody.

9. The isolated antibody of claim 8, wherein said monoclonal antibody is a human monoclonal antibody.

10. The isolated antibody of claim 7, wherein the antibody has Severe Acute Respiratory Syndrome Coronavirus (SARS-Co-V) neutralizing activity.

11. An isolated nucleic acid molecule encoding the antibody of claim 7.

12. A vector comprising at least one nucleic acid molecule of claim 6.

13. A host comprising at least one vector of claim 12.

14. The host of claim 13, wherein the host is a cell.

15. A medicament for the detection, prevention and/or treatment of a condition in a subject resulting from a Severe Acute Respiratory Syndrome Coronavirus (SARS-Co-V), wherein said medicament comprises the peptide of claim 1.

16. An immunogen for the detection, prevention and/or treatment of a condition in a subject resulting from a Severe Acute Respiratory Syndrome Coronavirus (SARS-Co-V), wherein said immunogen comprises the isolated peptide of claim 1.

17. A vaccine comprising the isolated peptide of claim 1.

18. A medicament for the detection, prevention and/or treatment of a condition in a subject resulting from a Severe Acute Respiratory Syndrome Coronavirus (SARS-Co-V), wherein said medicament comprises the isolated antibody of claim 7.

19. (canceled)

20. A diagnostic test method for determining the presence of antibodies recognizing Severe Acute Respiratory Syndrome Coronavirus (SARS-Co-V) in a sample, said method comprising the steps of:
contacting said sample with the isolated peptide of claim 1 and
determining whether antibodies in said sample bind to said peptide.

21. A diagnostic test method for determining the presence of a binding molecule of SARS-CoV in a sample, said method comprising the steps of:
contacting said sample with the isolated antibody of claim 7 and
determining whether the antibody binds to the binding molecule of SARS-CoV in said sample.

22. The diagnostic test method of claim 20, wherein the sample is a sample from a human subject potentially infected with a Severe Acute Respiratory Syndrome Coronavirus (SARS-Co-V).

23. An isolated polypeptide, said isolated polypeptide comprising at least one of the peptides of claim 1.

24. The isolated polypeptide of claim 23, wherein the isolated polypeptide comprises an amino acid sequence selected from the group consisting of SEQ ID NO:1-SEQ ID NO:9 and SEQ ID NO:10.

25. A nucleic acid molecule encoding the fusion protein or conjugate of claim 5.

26. An antibody, wherein said antibody is able to specifically recognize the fusion protein or conjugate of claim 5.

27. A vector comprising at least one nucleic acid molecule of claim 11.

28. A host comprising at least one vector of claim 27.

29. A medicament for detection, prevention and/or treatment of a condition in a subject resulting from a Severe Acute Respiratory Syndrome Coronavirus (SARS-Co-V), wherein said medicament comprises the fusion protein or conjugate of claim 5.

30. A medicament for detection, prevention and/or treatment of a condition in a subject resulting from a Severe Acute Respiratory Syndrome Coronavirus (SARS-Co-V), wherein said medicament comprises the isolated nucleic acid molecule of claim 11.

31. An immunogen for detection, prevention and/or treatment of a condition in a subject resulting from a Severe Acute Respiratory Syndrome Coronavirus (SARS-Co-V), wherein said immunogen comprises a fusion protein or conjugate of claim 5.

32. An immunogen for detection, prevention and/or treatment of a condition in a subject resulting from a Severe Acute Respiratory Syndrome Coronavirus (SARS-Co-V), wherein said immunogen comprises the isolated nucleic acid molecule of claim 11.

33. A composition for the detection, prevention and/or treatment of a condition in a subject resulting from a Severe Acute Respiratory Syndrome Coronavirus (SARS-Co-V), wherein said composition comprises the isolated nucleic acid molecule of claim 11.

34. A composition for the detection, prevention and/or treatment of a condition in a subject resulting from a Severe Acute Respiratory Syndrome Coronavirus (SARS-Co-V), wherein said composition comprises the fusion protein or conjugate of claim 5.

35. A composition for the detection, prevention and/or treatment of a condition in a subject resulting from a Severe Acute Respiratory Syndrome Coronavirus (SARS-Co-V), wherein said composition comprises the vector of claim 27.

36. A diagnostic test method for determining the presence of antibodies recognizing Severe Acute Respiratory Syndrome Coronavirus (SARS-Co-V) in a sample, said method comprising the steps of:
contacting said sample with the fusion protein or conjugate of claim 5 and
determining whether antibodies in said sample bind to said fusion protein or conjugate.

37. The diagnostic test method of claims 21, wherein the sample is a sample from a human subject believed to be infected with a Severe Acute Respiratory Syndrome Coronavirus (SARS-Co-V).


SEQUENCE LISTING (205 sequences)

SEQ ID NO: 1 - protein, 44 aa, SARS-CoV TOR2
MNELTLIDFYLCFLAFLLFLVLIMLIIFWFSLEIQDLEEPCTKV

SEQ ID NO: 2 - protein, 39 aa, SARS-CoV TOR2
MKLLIVLTCISLCSCICTVVQRCASNKPHVLEDPCKVQH

SEQ ID NO: 3 - protein, 98 aa, SARS-CoV TOR2
MDPNQTNVVPPALHLVDPQIQLTITRMEDAMGQGQNSADPKVYPIILRLGSQLSLSMARRNLDSLEARAFQSTPIVVQMTKLATTEELPDEFVVVTAK

SEQ ID NO: 4 - protein, 70 aa, SARS-CoV TOR2
MLPPCYNFLKEQHCQKASTQREAEAAVKPLLAPHHVVAVIQEIQLLAAVGEILLLEWLAEVVKLPSRYCC

SEQ ID NO: 5 - protein, 44 aa, SARS-CoV Frankfurt 1; identical to SEQ ID NO: 1

SEQ ID NO: 6 - protein, 98 aa, SARS-CoV Frankfurt 1; identical to SEQ ID NO: 3

SEQ ID NO: 7 - protein, 44 aa, SARS-CoV HSR 1; identical to SEQ ID NO: 1

SEQ ID NO: 8 - protein, 98 aa, SARS-CoV HSR 1; identical to SEQ ID NO: 3

SEQ ID NO: 9 - protein, 39 aa, SARS-CoV Frankfurt 1; identical to SEQ ID NO: 2

SEQ ID NO: 10 - protein, 39 aa, SARS-CoV HSR 1; identical to SEQ ID NO: 2

SEQ ID NO: 11 - protein, 15 aa, Artificial sequence
  Synthetic Peptide
LIMLIIFWFSLEIQD

SEQ ID NO: 12 - protein, 15 aa, Artificial sequence
  Synthetic Peptide
IMLIIFWFSLEIQDL

SEQ ID NO: 13 - protein, 15 aa, Artificial sequence
  Synthetic Peptide
MLIIFWFSLEIQDLE

SEQ ID NO: 14 - protein, 15 aa, Artificial sequence
  Synthetic Peptide
LIIFWFSLEIQDLEE

SEQ ID NO: 15 - protein, 15 aa, Artificial sequence
  Synthetic Peptide
IIFWFSLEIQDLEEP

SEQ ID NO: 16 - protein, 15 aa, Artificial sequence
  Synthetic Peptide
IFWFSLEIQDLEEPC

SEQ ID NO: 17 - protein, 15 aa, Artificial sequence
  Synthetic Peptide
FWFSLEIQDLEEPCT

SEQ ID NO: 18 - protein, 15 aa, Artificial sequence
  Synthetic Peptide
WFSLEIQDLEEPCTK

SEQ ID NO: 19 - protein, 15 aa, Artificial sequence
  Synthetic Peptide
FSLEIQDLEEPCTKV

SEQ ID NO: 20 - protein, 15 aa, Artificial sequence
  Synthetic Peptide
CSCICTVVQRCASNK

SEQ ID NO: 21 - protein, 15 aa, Artificial sequence
  Synthetic Peptide
SCICTVVQRCASNKP

SEQ ID NO: 22 - protein, 15 aa, Artificial sequence
  Synthetic Peptide
CICTVVQRCASNKPH

SEQ ID NO: 23 - protein, 15 aa, Artificial sequence
  Synthetic Peptide
ICTVVQRCASNKPHV

SEQ ID NO: 24 - protein, 15 aa, Artificial sequence
  Synthetic Peptide
CTVVQRCASNKPHVL

SEQ ID NO: 25 - protein, 15 aa, Artificial sequence
  Synthetic Peptide
TVVQRCASNKPHVLE

SEQ ID NO: 26 - protein, 15 aa, Artificial sequence
  Synthetic Peptide
VVQRCASNKPHVLED

SEQ ID NO: 27 - protein, 15 aa, Artificial sequence
  Synthetic Peptide
VQRCASNKPHVLEDP

SEQ ID NO: 28 - protein, 15 aa, Artificial sequence
  Synthetic Peptide
QRCASNKPHVLEDPC

SEQ ID NO: 29 - protein, 15 aa, Artificial sequence
  Synthetic Peptide
RCASNKPHVLEDPCK

SEQ ID NO: 30 - protein, 15 aa, Artificial sequence
  Synthetic Peptide
DPNQTNVVPPALHLV

SEQ ID NO: 31 - protein, 15 aa, Artificial sequence
  Synthetic Peptide
PNQTNVVPPALHLVD

SEQ ID NO: 32 - protein, 15 aa, Artificial sequence
  Synthetic Peptide
NQTNVVPPALHLVDP

SEQ ID NO: 33 - protein, 15 aa, Artificial sequence
  Synthetic Peptide
QTNVVPPALHLVDPQ

SEQ ID NO: 34 - protein, 15 aa, Artificial sequence
  Synthetic Peptide
TNVVPPALHLVDPQI

SEQ ID NO: 35 - protein, 15 aa, Artificial sequence
  Synthetic Peptide
NVVPPALHLVDPQIQ

SEQ ID NO: 36 - protein, 15 aa, Artificial sequence
  Synthetic Peptide
VVPPALHLVDPQIQL

SEQ ID NO: 37 - protein, 15 aa, Artificial sequence
  Synthetic Peptide
VPPALHLVDPQIQLT

SEQ ID NO: 38 - protein, 15 aa, Artificial sequence
  Synthetic Peptide
PPALHLVDPQIQLTI

SEQ ID NO: 39 - protein, 15 aa, Artificial sequence
  Synthetic Peptide
PALHLVDPQIQLTIT

SEQ ID NO: 40 - protein, 15 aa, Artificial sequence
  Synthetic Peptide
LTITRMEDAMGQGQN

SEQ ID NO: 41 - protein, 15 aa, Artificial sequence
  Synthetic Peptide
TITRMEDAMGQGQNS

SEQ ID NO: 42 - protein, 15 aa, Artificial sequence
  Synthetic Peptide
ITRMEDAMGQGQNSA

SEQ ID NO: 43 - protein, 15 aa, Artificial sequence
  Synthetic Peptide
TRMEDAMGQGQNSAD

SEQ ID NO: 44 - protein, 15 aa, Artificial sequence
  Synthetic Peptide
RMEDAMGQGQNSADP

SEQ ID NO: 45 - protein, 15 aa, Artificial sequence
  Synthetic Peptide
MEDAMGQGQNSADPK

SEQ ID NO: 46 - protein, 15 aa, Artificial sequence
  Synthetic Peptide
VVQMTKLATTEELPD

SEQ ID NO: 47 - protein, 15 aa, Artificial sequence
  Synthetic Peptide
VQMTKLATTEELPDE

SEQ ID NO: 48 - protein, 15 aa, Artificial sequence
  Synthetic Peptide
QMTKLATTEELPDEF

SEQ ID NO: 49 - protein, 15 aa, Artificial sequence
  Synthetic Peptide
MTKLATTEELPDEFV

SEQ ID NO: 50 - protein, 15 aa, Artificial sequence
  Synthetic Peptide
TKLATTEELPDEFVV

SEQ ID NO: 51 - protein, 15 aa, Artificial sequence
  Synthetic Peptide
KLATTEELPDEFVVV

SEQ ID NO: 52 - protein, 15 aa, Artificial sequence
  Synthetic Peptide
LATTEELPDEFVVVT

SEQ ID NO: 53 - protein, 15 aa, Artificial sequence
  Synthetic Peptide
ATTEELPDEFVVVTA

SEQ ID NO: 54 - protein, 15 aa, Artificial sequence
  Synthetic Peptide
LPPCYNFLKEQHCQK

SEQ ID NO: 55 - protein, 15 aa, Artificial sequence
  Synthetic Peptide
PPCYNFLKEQHCQKA

SEQ ID NO: 56 - protein, 15 aa, Artificial sequence
  Synthetic Peptide
PCYNFLKEQHCQKAS

SEQ ID NO: 57 - protein, 15 aa, Artificial sequence
  Synthetic Peptide
CYNFLKEQHCQKAST

SEQ ID NO: 58 - protein, 15 aa, Artificial sequence
  Synthetic Peptide
YNFLKEQHCQKASTQ

SEQ ID NO: 59 - protein, 15 aa, Artificial sequence
  Synthetic Peptide
NFLKEQHCQKASTQR

SEQ ID NO: 60 - protein, 15 aa, Artificial sequence
  Synthetic Peptide
FLKEQHCQKASTQRE

SEQ ID NO: 61 - protein, 15 aa, Artificial sequence
  Synthetic Peptide
LKEQHCQKASTQREA

SEQ ID NO: 62 - protein, 15 aa, Artificial sequence
  Synthetic Peptide
KEQHCQKASTQREAE

SEQ ID NO: 63 - protein, 15 aa, Artificial sequence
  Synthetic Peptide
EQHCQKASTQREAEA

SEQ ID NO: 64 - protein, 15 aa, Artificial sequence
  Synthetic Peptide
QHCQKASTQREAEAA

SEQ ID NO: 65 - protein, 15 aa, Artificial sequence
  Synthetic Peptide
HCQKASTQREAEAAV

SEQ ID NO: 66 - protein, 15 aa, Artificial sequence
  Synthetic Peptide
CQKASTQREAEAAVK

SEQ ID NO: 67 - protein, 15 aa, Artificial sequence
  Synthetic Peptide
QKASTQREAEAAVKP

SEQ ID NO: 68 - protein, 15 aa, Artificial sequence
  Synthetic Peptide
KASTQREAEAAVKPL

SEQ ID NO: 69 - protein, 15 aa, Artificial sequence
  Synthetic Peptide
ASTQREAEAAVKPLL

SEQ ID NO: 70 - protein, 15 aa, Artificial sequence
  Synthetic Peptide
STQREAEAAVKPLLA

SEQ ID NO: 71 - protein, 15 aa, Artificial sequence
  Synthetic Peptide
MNELTLIDFYLCFLA

SEQ ID NO: 72 - protein, 15 aa, Artificial sequence
  Synthetic Peptide
NELTLIDFYLCFLAF

SEQ ID NO: 73 - protein, 15 aa, Artificial sequence
  Synthetic Peptide
ELTLIDFYLCFLAFL

SEQ ID NO: 74 - protein, 15 aa, Artificial sequence
  Synthetic Peptide
LTLIDFYLCFLAFLL

SEQ ID NO: 75 - protein, 15 aa, Artificial sequence
  Synthetic Peptide
TLIDFYLCFLAFLLF

SEQ ID NO: 76 - protein, 15 aa, Artificial sequence
  Synthetic Peptide
LIDFYLCFLAFLLFL

SEQ ID NO: 77 - protein, 15 aa, Artificial sequence
  Synthetic Peptide
IDFYLCFLAFLLFLV

SEQ ID NO: 78 - protein, 15 aa, Artificial sequence
  Synthetic Peptide
DFYLCFLAFLLFLVL

SEQ ID NO: 79 - protein, 15 aa, Artificial sequence
  Synthetic Peptide
FYLCFLAFLLFLVLI

SEQ ID NO: 80 - protein, 15 aa, Artificial sequence
  Synthetic Peptide
YLCFLAFLLFLVLIM

SEQ ID NO: 81 - protein, 15 aa, Artificial sequence
  Synthetic Peptide
LCFLAFLLFLVLIML

SEQ ID NO: 82 - protein, 15 aa, Artificial sequence
  Synthetic Peptide
CFLAFLLFLVLIMLI

SEQ ID NO: 83 - protein, 15 aa, Artificial sequence
  Synthetic Peptide
FLAFLLFLVLIMLII

SEQ ID NO: 84 - protein, 15 aa, Artificial sequence
  Synthetic Peptide
LAFLLFLVLIMLIIF

SEQ ID NO: 85 - protein, 15 aa, Artificial sequence
  Synthetic Peptide
AFLLFLVLIMLIIFW

SEQ ID NO: 86 - protein, 15 aa, Artificial sequence
  Synthetic Peptide
FLLFLVLIMLIIFWF

SEQ ID NO: 87 - protein, 15 aa, Artificial sequence
  Synthetic Peptide
LLFLVLIMLIIFWFS

SEQ ID NO: 88 - protein, 15 aa, Artificial sequence
  Synthetic Peptide
LFLVLIMLIIFWFSL

SEQ ID NO: 89 - protein, 15 aa, Artificial sequence
  Synthetic Peptide
FLVLIMLIIFWFSLE

SEQ ID NO: 90 - protein, 15 aa, Artificial sequence
  Synthetic Peptide
LVLIMLIIFWFSLEI

SEQ ID NO: 91 - protein, 15 aa, Artificial sequence
  Synthetic Peptide
VLIMLIIFWFSLEIQ

SEQ ID NO: 92 - protein, 15 aa, Artificial sequence
  Synthetic Peptide
MKLLIVLTCISLCSC

SEQ ID NO: 93 - protein, 15 aa, Artificial sequence
  Synthetic Peptide
KLLIVLTCISLCSCI

SEQ ID NO: 94 - protein, 15 aa, Artificial sequence
  Synthetic Peptide
LLIVLTCISLCSCIC

SEQ ID NO: 95 - protein, 15 aa, Artificial sequence
  Synthetic Peptide
LIVLTCISLCSCICT

SEQ ID NO: 96 - protein, 15 aa, Artificial sequence
  Synthetic Peptide
IVLTCISLCSCICTV

SEQ ID NO: 97 - protein, 15 aa, Artificial sequence
  Synthetic Peptide
VLTCISLCSCICTVV

SEQ ID NO: 98 - protein, 15 aa, Artificial sequence
  Synthetic Peptide
LTCISLCSCICTVVQ

SEQ ID NO: 99 - protein, 15 aa, Artificial sequence
  Synthetic Peptide
TCISLCSCICTVVQR

SEQ ID NO: 100 - protein, 15 aa, Artificial sequence
  Synthetic Peptide
CISLCSCICTVVQRC

SEQ ID NO: 101 - protein, 15 aa, Artificial sequence
  Synthetic Peptide
ISLCSCICTVVQRCA

SEQ ID NO: 102 - protein, 15 aa, Artificial sequence
  Synthetic Peptide
SLCSCICTVVQRCAS

SEQ ID NO: 103 - protein, 15 aa, Artificial sequence
  Synthetic Peptide
LCSCICTVVQRCASN

SEQ ID NO: 104 - protein, 15 aa, Artificial sequence
  Synthetic Peptide
CASNKPHVLEDPCKV

SEQ ID NO: 105 - protein, 15 aa, Artificial sequence
  Synthetic Peptide
ASNKPHVLEDPCKVQ

SEQ ID NO: 106 - protein, 15 aa, Artificial sequence
  Synthetic Peptide
SNKPHVLEDPCKVQH

SEQ ID NO: 107 - protein, 15 aa, Artificial sequence
  Synthetic Peptide
MDPNQTNVVPPALHL

SEQ ID NO: 108 - protein, 15 aa, Artificial sequence
  Synthetic Peptide
ALHLVDPQIQLTITR

SEQ ID NO: 109 - protein, 15 aa, Artificial sequence
  Synthetic Peptide
LHLVDPQIQLTITRM

SEQ ID NO: 110 - protein, 15 aa, Artificial sequence
  Synthetic Peptide
HLVDPQIQLTITRME

SEQ ID NO: 111 - protein, 15 aa, Artificial sequence
  Synthetic Peptide
LVDPQIQLTITRMED

SEQ ID NO: 112 - protein, 15 aa, Artificial sequence
  Synthetic Peptide
VDPQIQLTITRMEDA

SEQ ID NO: 113 - protein, 15 aa, Artificial sequence
  Synthetic Peptide
DPQIQLTITRMEDAM

SEQ ID NO: 114 - protein, 15 aa, Artificial sequence
  Synthetic Peptide
PQIQLTITRMEDAMG

SEQ ID NO: 115 - protein, 15 aa, Artificial sequence
  Synthetic Peptide
QIQLTITRMEDAMGQ

SEQ ID NO: 116 - protein, 15 aa, Artificial sequence
  Synthetic Peptide
IQLTITRMEDAMGQG

SEQ ID NO: 117 - protein, 15 aa, Artificial sequence
  Synthetic Peptide
QLTITRMEDAMGQGQ

SEQ ID NO: 118 - protein, 15 aa, Artificial sequence
  Synthetic Peptide
EDAMGQGQNSADPKV

SEQ ID NO: 119 - protein, 15 aa, Artificial sequence
  Synthetic Peptide
DAMGQGQNSADPKVY

SEQ ID NO: 120 - protein, 15 aa, Artificial sequence
  Synthetic Peptide
AMGQGQNSADPKVYP

SEQ ID NO: 121 - protein, 15 aa, Artificial sequence
  Synthetic Peptide
MGQGQNSADPKVYPI

SEQ ID NO: 122 - protein, 15 aa, Artificial sequence
  Synthetic Peptide
GQGQNSADPKVYPII

SEQ ID NO: 123 - protein, 15 aa, Artificial sequence
  Synthetic Peptide
QGQNSADPKVYPIIL

SEQ ID NO: 124 - protein, 15 aa, Artificial sequence
  Synthetic Peptide
GQNSADPKVYPIILR

SEQ ID NO: 125 - protein, 15 aa, Artificial sequence
  Synthetic Peptide
QNSADPKVYPIILRL

SEQ ID NO: 126 - protein, 15 aa, Artificial sequence
  Synthetic Peptide
NSADPKVYPIILRLG

SEQ ID NO: 127 - protein, 15 aa, Artificial sequence
  Synthetic Peptide
SADPKVYPIILRLGS

SEQ ID NO: 128 - protein, 15 aa, Artificial sequence
  Synthetic Peptide
ADPKVYPIILRLGSQ

SEQ ID NO: 129 - protein, 15 aa, Artificial sequence
  Synthetic Peptide
DPKVYPIILRLGSQL

SEQ ID NO: 130 - protein, 15 aa, Artificial sequence
  Synthetic Peptide
PKVYPIILRLGSQLS

SEQ ID NO: 131 - protein, 15 aa, Artificial sequence
  Synthetic Peptide
KVYPIILRLGSQLSL

SEQ ID NO: 132 - protein, 15 aa, Artificial sequence
  Synthetic Peptide
VYPIILRLGSQLSLS

SEQ ID NO: 133 - protein, 15 aa, Artificial sequence
  Synthetic Peptide
YPIILRLGSQLSLSM

SEQ ID NO: 134 - protein, 15 aa, Artificial sequence
  Synthetic Peptide
PIILRLGSQLSLSMA

SEQ ID NO: 135 - protein, 15 aa, Artificial sequence
  Synthetic Peptide
IILRLGSQLSLSMAR

SEQ ID NO: 136 - protein, 15 aa, Artificial sequence
  Synthetic Peptide
ILRLGSQLSLSMARR

SEQ ID NO: 137 - protein, 15 aa, Artificial sequence
  Synthetic Peptide
LRLGSQLSLSMARRN

SEQ ID NO: 138 - protein, 15 aa, Artificial sequence
  Synthetic Peptide
RLGSQLSLSMARRNL

SEQ ID NO: 139 - protein, 15 aa, Artificial sequence
  Synthetic Peptide
LGSQLSLSMARRNLD

SEQ ID NO: 140 - protein, 15 aa, Artificial sequence
  Synthetic Peptide
GSQLSLSMARRNLDS

SEQ ID NO: 141 - protein, 15 aa, Artificial sequence
  Synthetic Peptide
SQLSLSMARRNLDSL

SEQ ID NO: 142 - protein, 15 aa, Artificial sequence
  Synthetic Peptide
QLSLSMARRNLDSLE

SEQ ID NO: 143 - protein, 15 aa, Artificial sequence
  Synthetic Peptide
LSLSMARRNLDSLEA

SEQ ID NO: 144 - protein, 15 aa, Artificial sequence
  Synthetic Peptide
SLSMARRNLDSLEAR

SEQ ID NO: 145 - protein, 15 aa, Artificial sequence
  Synthetic Peptide
LSMARRNLDSLEARA

SEQ ID NO: 146 - protein, 15 aa, Artificial sequence
  Synthetic Peptide
SMARRNLDSLEARAF

SEQ ID NO: 147 - protein, 15 aa, Artificial sequence
  Synthetic Peptide
MARRNLDSLEARAFQ

SEQ ID NO: 148 - protein, 15 aa, Artificial sequence
  Synthetic Peptide
ARRNLDSLEARAFQS

SEQ ID NO: 149 - protein, 15 aa, Artificial sequence
  Synthetic Peptide
RRNLDSLEARAFQST

SEQ ID NO: 150 - protein, 15 aa, Artificial sequence
  Synthetic Peptide
RNLDSLEARAFQSTP

SEQ ID NO: 151 - protein, 15 aa, Artificial sequence
  Synthetic Peptide
NLDSLEARAFQSTPI

SEQ ID NO: 152 - protein, 15 aa, Artificial sequence
  Synthetic Peptide
LDSLEARAFQSTPIV

SEQ ID NO: 153 - protein, 15 aa, Artificial sequence
  Synthetic Peptide
DSLEARAFQSTPIVV

SEQ ID NO: 154 - protein, 15 aa, Artificial sequence
  Synthetic Peptide
SLEARAFQSTPIVVQ

SEQ ID NO: 155 - protein, 15 aa, Artificial sequence
  Synthetic Peptide
LEARAFQSTPIVVQM

SEQ ID NO: 156 - protein, 15 aa, Artificial sequence
  Synthetic Peptide
EARAFQSTPIVVQMT

SEQ ID NO: 157 - protein, 15 aa, Artificial sequence
  Synthetic Peptide
ARAFQSTPIVVQMTK

SEQ ID NO: 158 - protein, 15 aa, Artificial sequence
  Synthetic Peptide
RAFQSTPIVVQMTKL

SEQ ID NO: 159 - protein, 15 aa, Artificial sequence
  Synthetic Peptide
AFQSTPIVVQMTKLA

SEQ ID NO: 160 - protein, 15 aa, Artificial sequence
  Synthetic Peptide
FQSTPIVVQMTKLAT

SEQ ID NO: 161 - protein, 15 aa, Artificial sequence
  Synthetic Peptide
QSTPIVVQMTKLATT

SEQ ID NO: 162 - protein, 15 aa, Artificial sequence
  Synthetic Peptide
STPIVVQMTKLATTE

SEQ ID NO: 163 - protein, 15 aa, Artificial sequence
  Synthetic Peptide
TPIVVQMTKLATTEE

SEQ ID NO: 164 - protein, 15 aa, Artificial sequence
  Synthetic Peptide
PIVVQMTKLATTEEL

SEQ ID NO: 165 - protein, 15 aa, Artificial sequence
  Synthetic Peptide
IVVQMTKLATTEELP

SEQ ID NO: 166 - protein, 15 aa, Artificial sequence
  Synthetic Peptide
TTEELPDEFVVVTAK

SEQ ID NO: 167 - protein, 15 aa, Artificial sequence
  Synthetic Peptide
MLPPCYNFLKEQHCQ

SEQ ID NO: 168 - protein, 15 aa, Artificial sequence
  Synthetic Peptide
TQREAEAAVKPLLAP

SEQ ID NO: 169 - protein, 15 aa, Artificial sequence
  Synthetic Peptide
QREAEAAVKPLLAPH

SEQ ID NO: 170 - protein, 15 aa, Artificial sequence
  Synthetic Peptide
REAEAAVKPLLAPHH

SEQ ID NO: 171 - protein, 15 aa, Artificial sequence
  Synthetic Peptide
EAEAAVKPLLAPHHV

SEQ ID NO: 172 - protein, 15 aa, Artificial sequence
  Synthetic Peptide
AEAAVKPLLAPHHVV

SEQ ID NO: 173 - protein, 15 aa, Artificial sequence
  Synthetic Peptide
EAAVKPLLAPHHVVA

SEQ ID NO: 174 - protein, 15 aa, Artificial sequence
  Synthetic Peptide
AAVKPLLAPHHVVAV

SEQ ID NO: 175 - protein, 15 aa, Artificial sequence
  Synthetic Peptide
AVKPLLAPHHVVAVI

SEQ ID NO: 176 - protein, 15 aa, Artificial sequence
  Synthetic Peptide
VKPLLAPHHVVAVIQ

SEQ ID NO: 177 - protein, 15 aa, Artificial sequence
  Synthetic Peptide
KPLLAPHHVVAVIQE

SEQ ID NO: 178 - protein, 15 aa, Artificial sequence
  Synthetic Peptide
PLLAPHHVVAVIQEI

SEQ ID NO: 179 - protein, 15 aa, Artificial sequence
  Synthetic Peptide
LLAPHHVVAVIQEIQ

SEQ ID NO: 180 - protein, 15 aa, Artificial sequence
  Synthetic Peptide
LAPHHVVAVIQEIQL

SEQ ID NO: 181 - protein, 15 aa, Artificial sequence
  Synthetic Peptide
APHHVVAVIQEIQLL

SEQ ID NO: 182 - protein, 15 aa, Artificial sequence
  Synthetic Peptide
PHHVVAVIQEIQLLA

SEQ ID NO: 183 - protein, 15 aa, Artificial sequence
  Synthetic Peptide
HHVVAVIQEIQLLAA

SEQ ID NO: 184 - protein, 15 aa, Artificial sequence
  Synthetic Peptide
HVVAVIQEIQLLAAV

SEQ ID NO: 185 - protein, 15 aa, Artificial sequence
  Synthetic Peptide
VVAVIQEIQLLAAVG

SEQ ID NO: 186 - protein, 15 aa, Artificial sequence
  Synthetic Peptide
VAVIQEIQLLAAVGE

SEQ ID NO: 187 - protein, 15 aa, Artificial sequence
  Synthetic Peptide
AVIQEIQLLAAVGEI

SEQ ID NO: 188 - protein, 15 aa, Artificial sequence
  Synthetic Peptide
VIQEIQLLAAVGEIL

SEQ ID NO: 189 - protein, 15 aa, Artificial sequence
  Synthetic Peptide
IQEIQLLAAVGEILL

SEQ ID NO: 190 - protein, 15 aa, Artificial sequence
  Synthetic Peptide
QEIQLLAAVGEILLL

SEQ ID NO: 191 - protein, 15 aa, Artificial sequence
  Synthetic Peptide
EIQLLAAVGEILLLE

SEQ ID NO: 192 - protein, 15 aa, Artificial sequence
  Synthetic Peptide
IQLLAAVGEILLLEW

SEQ ID NO: 193 - protein, 15 aa, Artificial sequence
  Synthetic Peptide
QLLAAVGEILLLEWL

SEQ ID NO: 194 - protein, 15 aa, Artificial sequence
  Synthetic Peptide
LLAAVGEILLLEWLA

SEQ ID NO: 195 - protein, 15 aa, Artificial sequence
  Synthetic Peptide
LAAVGEILLLEWLAE

SEQ ID NO: 196 - protein, 15 aa, Artificial sequence
  Synthetic Peptide
AAVGEILLLEWLAEV

SEQ ID NO: 197 - protein, 15 aa, Artificial sequence
  Synthetic Peptide
AVGEILLLEWLAEVV

SEQ ID NO: 198 - protein, 15 aa, Artificial sequence
  Synthetic Peptide
VGEILLLEWLAEVVK

SEQ ID NO: 199 - protein, 15 aa, Artificial sequence
  Synthetic Peptide
GEILLLEWLAEVVKL

SEQ ID NO: 200 - protein, 15 aa, Artificial sequence
  Synthetic Peptide
EILLLEWLAEVVKLP

SEQ ID NO: 201 - protein, 15 aa, Artificial sequence
  Synthetic Peptide
ILLLEWLAEVVKLPS

SEQ ID NO: 202 - protein, 15 aa, Artificial sequence
  Synthetic Peptide
LLLEWLAEVVKLPSR

SEQ ID NO: 203 - protein, 15 aa, Artificial sequence
  Synthetic Peptide
LLEWLAEVVKLPSRY

SEQ ID NO: 204 - protein, 15 aa, Artificial sequence
  Synthetic Peptide
LEWLAEVVKLPSRYC

SEQ ID NO: 205 - protein, 15 aa, Artificial sequence
  Synthetic Peptide
EWLAEVVKLPSRYCC